SEQUENCE LISTING (10 sequences)

SEQ ID NO: 1 - DNA, 23 bp, Mus musculus
gcaggatctcctgtcatctcacc

SEQ ID NO: 2 - DNA, 23 bp, Mus musculus
gatgctcttcgtccagatcatcc

SEQ ID NO: 3 - DNA, 24 bp, Mus musculus
gaggtagaaacaagagaatcatgg

SEQ ID NO: 4 - DNA, 21 bp, Mus musculus
gttcgcactgctgtatgttgc

SEQ ID NO: 5 - DNA, 22 bp, Mus musculus
agtccctctggccatgccatgg

SEQ ID NO: 6 - DNA, 25 bp, Mus musculus
agggcaagatcatcatgcacgaccc

SEQ ID NO: 7 - DNA, 19 bp, Mus musculus
cacctgtgagaccggacca

SEQ ID NO: 8 - DNA, 27 bp, Mus musculus
gaccataacatagtatacacctgctgc

SEQ ID NO: 9 - DNA, 27 bp, Mus musculus
gaccataacatagtatacacctgctgc

SEQ ID NO: 10 - DNA, 18 bp, Mus musculus
agaagggtcgcatggcaa

(19) United States
(12) Patent Application Publication
(10) Pub. No.: US 2003/0157076 A1
(43) Pub. Date: Aug. 21, 2003

(54) Disruption of the Akt2 gene

(75) Inventors: Kevin G. Coleman, Old Lyme, CT (US); Robert S. Garofalo, East Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/360,203

(22) Filed: Feb. 7, 2003

Related U.S. Application Data
(60) Provisional application No. 60/355,106, filed on Feb. 8, 2002.

(51) Int. Cl. C12Q 1/68; A01K 67/027; A61K 48/00; C12N 5/06; C12N 5/08

(52) U.S. Cl. 424/93.21; 800/18; 435/6; 435/354; 435/366

(57) ABSTRACT

The invention features non-human mammals and animal cells that contain a targeted disruption of an Akt2 gene.

This application claims priority, under 35 U.S.C. § 119(e), from provisional application 60/355,106, filed Feb. 8, 2002.

### FIELD OF THE INVENTION

The present invention features genetically-modified non-human mammals and animal cells containing a disrupted serine threonine kinase Akt2 gene (also known as the PKBβ gene).

### BACKGROUND OF THE INVENTION

Akt is the cellular homologue of the transforming oncogene of the AKT8 oncovirus (v-Akt) (Bellacosa et al., Science 254: 274-77, 1991). In mammals, there are three Akt genes, which yield gene products that are similar in structure and size (Altomare et al., Cytogenet. Cell Genet. 74: 248, 1996; Bellacosa et al., Oncogene 8: 745, 1993; Murthy et al., Cygenet. Cell Genet. 88: 38, 2000). The Akts fall into the AGC (protein kinase A/protein kinase G/protein kinase C-like) class of protein kinases and require phosphorylation for activation (Scheid and Woodgett, Nature Reviews 2: 760-768, 2001).

Akts are positively regulated by phosphatidyl inositol 3-kinase (PI3-K) activation and their substrates include glycogen synthase kinase-3 (GSK-3), 6-phosphofructo-2-kinase, the pro-apoptotic protein BAD, various transcription factors (FKHR, FKHRL1, and AFX), p21^{Cip1}, RAF, endothelial nitric oxide synthase, mTOR, BRCA1, and some I-κB kinases (Cohen and Frame, Nature Reviews 2: 769-76, 2001; Deprez et al., J. Biol. Chem. 272: 17269-75, 1997; Datta et al., Cell 91: 231-41, 1997; Brunet et al., Cell 96: 857-68, 1999; Biggs et al., Proc. Natl. Acad. Sci. USA 96: 7421-26, 1999; Kops et al., Nature 398: 630-34, 1999; Rossig et al., Mol. Cell. Biol. 21: 5644-57, 2001; Zimmerman and Moelling, Science 286: 1741-44, 1999; Rommel et al., Science 286: 1738-41, 1999; Guan et al., J. Biol. Chem. 275: 27354-59, 2000; Michell et al., Curr. Biol. 9: 845-48, 1999; Dimmeler et al., Nature 399: 601-05, 1999; Fulton et al., Nature 399: 597-601, 1999; Scott et al., Proc. Natl. Acad. Sci. USA 95: 7772-77, 1999; Nave et al., Biochem. J. 344: 427-31, 1999; Altiok et al., J. Biol. Chem. 274: 32274-78, 1999; Ozes et al., Nature 401: 82-85, 1998; Romashikova and Makarov, Nature 401: 86-90, 1999). Thus, Akt function is clearly implicated as playing a role in regulating metabolism, transcription, protein translation, cell growth, and cell survival (Scheid and Wood, supra), and may play a role, via GSK-3, in the hyperphosphorylation of tau and the development of Alzheimer's Disease (Hanger et al., Neurosci. Lett. 147: 58-62, 1992; Mandelkow et al., FEBS Lett. 314: 315-21, 1992; Takashima et al., Proc. Natl. Acad. Sci. USA 90: 7789-93, 1993).

Akt2 is the Akt homologue most highly expessed in insulin-sensitive tissues, and it has been suggested that Akt2 plays a role in insulin signaling (Calera et al., J. Biol. Chem. 273: 7201, 1998; Hill et al., Mol. Cell. Biol. 19: 7771, 1999; Sumers et al., J. Biol. Chem. 274: 23858, 1999). However, additional research tools, including Akt2 knockout mice, would be useful to further define the physiological role of Akt2 action, and the therapeutic implications associated with modulating Akt2 activity.

### SUMMARY OF THE INVENTION

The invention features a genetically-modified, non-human mammal, wherein the modification results in a disrupted Akt2 gene in the mammal's genome. Preferably, the mammal exhibits lipoatrophy, insulin resistance, glucose intolerance, reduced body weight, hyperglycemia, or corneal degeneration, and the mammal is a rodent, more preferably, a mouse. Preferably, the mammal is homozygous for the modification.

In another aspect, the invention features a genetically-modified animal cell, wherein the modification comprises a disrupted Akt2 gene. Preferably, the cell is an embryonic stem (ES) cell or an ES-like cell, the cell is murine or human, or the cell is homozygous for the modification. In another preferred embodiment, the cell is isolated from a genetically-modified, non-human mammal containing a modification that results in a disrupted Akt2 gene, preferably, the cell is an embryonic fibroblast, stem cell, neuron, skeletal or cardiac muscle cell, myoblast, brown or white adipocyte, hepatocyte, or pancreatic β cell.

Also featured is a method of identifying a therapeutic agent for diabetes, comprising administering an agent to a genetically-modified mouse homozygous for a disrupted Akt2 gene and assessing a diabetic phenotype in the mammal, wherein the agent is identified as a treatment for diabetes if the mammal demonstrates an improvement in the diabetic phenotype. Preferably, the improved diabetic phenotype is reduced plasma glucose or increased insulin sensitivity.

In another aspect, the invention provides a method of identifying a gene that demonstrates modified expression as a result of reduced Akt2 activity in an animal cell, comprising assessing the expression profile of at least one gene other than Akt2 in an animal cell homozygous for a genetic modification that disrupts an Akt2 gene, and comparing the profile to that from a wild type cell.

In a related aspect, the invention features a method of identifying a protein that demonstrates a modified level or post-translational processing as a result of reduced Akt2 activity in an animal cell comprising comparing the level or post-translational characteristics of the protein in an animal cell homozygous for a genetic modification that disrupts the Akt2 gene to the level or post-translational characteristics of the protein in an appropriate wild-type control.

In addition, the invention provides a method of identifying a biological characteristic associated with reduction or elimination of Akt2 activity comprising comparing a biological characteristic of a genetically-modified mouse homozygous for a genetic modification that disrupts the Akt2 gene, or a genetically-modified animal cell homozygous for a genetic modification that disrupts the Akt2 gene, to the characteristic of the appropriate wild-type control.

Those skilled in the art will fully understand the terms used herein in the description and the appendant claims to describe the present invention. Nonetheless, unless otherwise provided herein, the following terms are as described immediately below.

A non-human mammal or an animal cell that is “genetically-modified” is heterozygous or homozygous for a modification that is introduced into the non-human mammal or animal cell, or into a progenitor non-human mammal or animal cell, by genetic engineering. The standard methods of genetic engineering that are available for introducing the modification include homologous recombination, viral vector gene trapping, irradiation, chemical mutagenesis, and the transgenic expression of a nucleotide sequence encoding antisense RNA alone or in combination with catalytic ribozymes. Preferred methods for genetic modification to disrupt a gene are those which modify an endogenous gene by inserting a “foreign nucleic acid sequence” into the gene locus, e.g., by homologous recombination or viral vector gene trapping. A “foreign nucleic acid sequence” is an exogenous sequence that is non-naturally occurring in the gene. This insertion of foreign DNA can occur within any region of the Akt2 gene, e.g., in an enhancer, promoter, regulator region, noncoding region, coding region, intron, or exon. The most preferred method of genetic engineering for gene disruption is homologous recombination, in which the foreign nucleic acid sequence is inserted in a targeted manner either alone or in combination with a deletion of a portion of the endogenous gene sequence.

By an Akt2 gene that is “disrupted” is meant an Akt2 gene that is genetically modified such that the cellular activity of the Akt2 polypeptide encoded by the disrupted gene is decreased or eliminated in cells that normally express a wild type version of the Akt2 gene. When the genetic modification effectively eliminates all wild type copies of the Akt2 gene in a cell (e.g., the genetically-modified, non-human mammal or animal cell is homozygous for the Akt2 gene disruption or the only wild type copy of the Akt2 gene originally present is now disrupted), the genetic modification results in a reduction in Akt2 polypeptide activity as compared to a control cell that expresses the wild type Akt2 gene. This reduction in Akt2 polypeptide activity results from either reduced Akt2 gene expression (i.e., Akt2 mRNA levels are effectively reduced resulting in reduced levels of Akt2 polypeptide) and/or because the disrupted Akt2 gene encodes a mutated polypeptide with altered, e.g., reduced, function as compared to a wild type Akt2 polypeptide. Preferably, the activity of Akt2 polypeptide in the genetically-modified, non-human mammal or animal cell is reduced to 50% or less of wild type levels, more preferably, to 25% or less, and, even more preferably, to 10% or less of wild type levels. Most preferably, the Akt2 gene disruption results in non-detectable Akt2 activity.

By a “genetically-modified, non-human mammal” containing a disrupted Akt2 gene is meant a non-human mammal that is originally produced, for example, by creating a blastocyst or embryo carrying the desired genetic modification and then implanting the blastocyst or embryo in a foster mother for in utero development. The genetically-modified blastocyst or embryo can be made, in the case of mice, by implanting a genetically-modified embryonic stem (ES) cell into a mouse blastocyst or by aggregating ES cells with tetraploid embryos. Alternatively, various species of genetically-modified embryos can be obtained by nuclear transfer. In the case of nuclear transfer, the donor cell is a somatic cell or a pluripotent stem cell, and it is engineered to contain the desired genetic modification that disrupts the Akt2 gene. The nucleus of this cell is then transferred into a fertilized or parthenogenetic oocyte that is enucleated; the resultant embryo is reconstituted and developed into a blastocyst. A genetically-modified blastocyst produced by either of the above methods is then implanted into a foster mother according to standard methods well known to those skilled in the art. A “genetically-modified, non-human mammal” includes all progeny of the non-human mammals created by the methods described above, provided that the progeny inherit at least one copy of the genetic modification that disrupts the Akt2 gene. It is preferred that all somatic cells and germline cells of the genetically-modified non-human mammal contain the modification. Preferred non-human mammals that are genetically-modified to contain a disrupted Akt2 gene include rodents, such as mice and rats, cats, dogs, rabbits, guinea pigs, hamsters, sheep, pigs, and ferrets.

By a “genetically-modified animal cell” containing a disrupted Akt2 gene is meant an animal cell, including a human cell, created by genetic engineering to contain a disrupted Akt2 gene, as well as daughter cells that inherit the disrupted Akt2 gene. These cells may be genetically-modified in culture according to any standard method known in the art. As an alternative to genetically modifying the cells in culture, non-human mammalian cells may also be isolated from a genetically-modified, non-human mammal that contains an Akt2 gene disruption. The animal cells of the invention may be obtained from primary cell or tissue preparations as well as culture-adapted, tumorigenic, or transformed cell lines. These cells and cell lines are derived, for example, from endothelial cells, epithelial cells, islets, neurons and other neural tissue-derived cells, mesothelial cells, osteocytes, lymphocytes, chondrocytes, hematopoietic cells, immune cells, cells of the major glands or organs (e.g., testicle, liver, lung, heart, stomach, pancreas; kidney, and skin), muscle cells (including cells from skeletal muscle, smooth muscle, and cardiac muscle), exocrine or endocrine cells, fibroblasts, and embryonic and other totipotent or pluripotent stem cells (e.g., ES cells, ES-like cells, and embryonic germline (EG) cells, and other stem cells, such as progenitor cells anid tissue-derived stem cells). The preferred genetically-modified cells are ES cells, more preferably, mouse or rat ES cells, and, most preferably, human ES cells.

By “reduced Akt2 activity” is meant a decrease in the activity of the Akt2 enzyme as a result of genetic manipulation of the Akt2 gene that causes a reduction in the level of functional Akt2 polypeptide in a cell, or as the result of administration of a pharmacological agent that inhibits Akt2 activity.

By an “ES cell” or an “ES-like cell” is meant a pluripotent stem cell derived from an embryo, from a primordial germ cell, or from a teratocarcinoma, that is capable of indefinite self renewal as well as differentiation into cell types that are representative of all three embryonic germ layers.

A “microarray” or “DNA array” means an arrangement of distinct polynucleotides on a substrate, as more fully described herein.

Other features and advantages of the invention will be apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. Additional guidance with respect to making and using nucleic acids and polypeptides is found in standard textbooks of molecular biology, protein science, and immunology (see, e.g., Davis et al., Basic Methods in Molecular Biology, Elsevir Sciences Publishing, Inc., New York, N.Y., 1986; Hames et al., Nucleic Acid Hybridization, IL Press, 1985; Molecular Cloning, Sambrook et al., Current Protocols in Molecular Biology, Eds. Ausubel et al., John Wiley and Sons; Current Protocols in Human Genetics, Eds. Dracopoli et al., John Wiley and Sons; Current Protocols in Protein Science, Eds. John E. Coligan et al., John Wiley and Sons; and Current Protocols in immunology, Eds. John E. Coligan et al., John Wiley and Sons). All publications mentioned herein are incorporated by reference in their entireties.

### BRIEF DESCRIPTION OF THE FIGURES

FIG. 1 is a schematic depicting the Akt2 gene targeting vector, the location for homologous recombination of the vector in the endogenous murine Akt2 gene, and the polymerase chain reaction (PCR) strategy used to verify gene targeting.

FIG. 2 shows the Southern analysis of ES Cells. Clone #2 contains the targeted allele as well as the endogenous allele.

FIG. 3 shows the results of polymerase chain reaction (PCR)-based genotyping of heterozygote (±) and knockout (−/−) mice with respect to the disrupted Akt2 allele.

FIG. 4A shows growth retardation in Akt2 KO mice. Body weights in Akt2 KO male (open circles) and female (open diamonds) mice were measured alongside age-matched wild type DBA/1lacj males (closed circles) and females (closed diamonds). (p<0.01 between wild type and Akt2 KO mice at all ages, n=9−13 mice per group.) FIG. 4B shows Akt2 KO growth retardation with respect to naso-anal length in Akt2 KO (open squares) mice as compared to wild-type. (*, p<0.01, n=5−12 mice per group.)

FIG. 5A shows adipose tissue mass of wild type and Akt2 KO female mice. FIG. 5B shows adipose tissue mass of wild type and Akt2 KO male mice. Adipose tissue mass was determined by Micro CT scanning in four regional depots, the inguinal subcutaneous (Ing), epididymal/gonadal, retroperitoneal (RP) and mesenteric (Mes) regions. Adipose tissue mass was significantly (p<0.05) reduced in both female (FIG. 5A) and male (FIG. 5B) Akt2 KO mice. In female Akt2 KO mice adipose depot mass was reduced 80-90% in all depots measured. In male Akt2 KO mice adipose depot mass was reduced 65-75% in the inguinal and epididymal depots, and >95% in the retroperitoneal and mesenteric depots.

FIG. 6 shows seven week old Akt2 KO mice exhibit fasting hyperglycemia and glucose intolerance in an oral glucose tolerance test. Blood samples were taken from overnight fasted Akt2 KO (open symbols) and wild type (closed symbols) mice at time zero. Mice were immediately given an oral dose of glucose (1 gm/kg) and blood sampled at the indicated times. Plasma glucose levels were significantly elevated in both male and female Akt2 KO mice (open circles and diamonds, respectively) relative to wild type male and female mice (closed circles and diamonds, respectively) at time zero and 30 minutes following the glucose load.

FIG. 7A shows hyperglycemia in male and female Akt2 KO mice. FIG. 7B shows hyperinsulinemia in Akt2 KO mice. Plasma glucose and insulin levels were determined every 14 days in male wild type (closed circles, n=11), male Akt2 KO (open circles, n=12), female wild type (closed diamonds, n=9) and female Akt2 KO (open diamonds, n=13) mice.

FIG. 8A shows plasma glucose (closed symbols) and insulin (open symbols) levels in 3 male Akt2 KO mice exhibiting β-cell failure. FIG. 8B shows the remaining nine male Akt2 KO mice, which were mildly hyperglycemic (closed diamonds) while becoming increasingly hyperinsulinemic (open squares), or insulin resistant, with age.

FIG. 9A shows that male Akt2 KO mice became severely hypoinsulinemic by 8 months of age. FIG. 9B shows that male Akt2 KO became severely hyperglycemic by 8 months of age.

FIG. 10 shows that muscle glucose uptake is impaired in Akt2 KO mice. 2-deoxyglucose (2-DG) uptake into isolated soleus muscles from male control and Akt2 KO mice was determined in the absence of insulin (basal, open bars) or in the presence of a submaximal (1 nM, hatched bars) or maximal (100 nM, filled bars) concentration of insulin. (*p<0.05 vs corresponding basal; **p<0.01 vs corresponding basal; #p<0.05 vs corresponding insulin-treated control sample.)

### DETAILED DESCRIPTION OF THE INVENTION

Genetically-Modified Non-human Mammals and Animal Cells Containing a Disrupted Akt2 Gene

1. Genetically-Modified Non-human Mammals and Animal Cells

The-genetically-modified, non-human mammals and genetically-modified animal cells, including human cells, of the invention are heterozygous or homozygous for a modification that disrupts the Akt2 gene. The animal cells may be derived by genetically engineering cells in culture, or, in the case of non-human mammalian cells, the cells may be isolated from genetically-modified, non-human mammals.

The Akt2 gene locus is disrupted by one of the several techniques for genetic modification known in the art, including chemical mutagenesis (Rinchik, Trends in Genetics 7: 15-21, 1991, Russell, Environmental & Molecular Mutagenesis 23 (Suppl. 24): 23-29, 1994), irradiation (Russell, supra), transgenic expression of Akt2 gene antisense RNA, either alone or in combination with a catalytic RNA ribozyme sequence (Luyckx et al., Proc. Natl. Acad. Sci. 96: 12174-79, 1999; Sokol et al., Transgenic Research 5: 363-71, 1996; Efrat et al., Proc. Natl. Acad. Sci. USA 91: 2051-55, 1994; Larsson et al., Nucleic Acids Research 22: 2242-48, 1994) and, as further discussed below, the disruption of the Akt2 gene by the insertion of a foreign nucleic acid sequence into the Akt2 gene locus. Preferably, the foreign sequence is inserted by homologous recombination or by the insertion of a viral vector. Most preferably, the method of Akt2 gene disruption to create the genetically modified non-human mammals and animal cells of the invention is homologous recombination and includes a deletion of a portion of the endogenous Akt2 gene sequence.

The integration of the foreign sequence disrupts the Akt2 gene through one or more of the following mechanisms: by interfering with the Akt2 gene transcription or translation process (e.g., by interfering with promoter recognition, or by introducing a transcription termination site or a translational stop codon into the Akt2 gene); or by distorting the Akt2 gene coding sequence such that it no longer encodes an Akt2 polypeptide with normal function (e.g., by inserting a foreign coding sequence into the Akt2 gene coding sequence, by introducing a frameshift mutation or amino acid(s) substitution, or, in the case of a double crossover event, by deleting a portion of the Akt2 gene coding sequence that is required for expression of a functional Akt2 protein).

To insert a foreign sequence into an Akt2 gene locus in the genome of a cell to create the genetically modified non-human mammals and animal cells of the invention based upon the present description, the foreign DNA sequence is introduced into the cell according to a standard method known in the art such as electroporation, calcium-phosphate precipitation, retroviral infection, microinjection, biolistics, liposome transfection, DEAE-dextran transfection, or transferrinfection (see, e.g., Neumann et al., EMBO J. 1: 841-845, 1982; Potter et al., Proc. Natl. Acad. Sci USA 81: 7161-65, 1984; Chu et al., Nucleic Acids Res. 15: 1311-26, 1987; Thomas and Capecchi, Cell 51: 503-12, 1987; Baum et al., Biotechniques 17: 1058-62, 1994; Biewenga et al., J. Neuroscience Methods 71: 67-75, 1997; Zhang et al., Biotechniques 15: 868-72, 1993; Ray and Gage, Biotechniques 13: 598-603, 1992; Lo, Mol. Cell. Biol. 3: 1803-14, 1983; Nickoloff et al., Mol. Biotech. 10: 93-101, 1998; Linney et al., Dev. Biol. (Orlando) 213: 207-16, 1999; Zimmer and Gruss, Nature 338: 150-153, 1989; and Robertson et al., Nature 323: 445-48, 1986). The preferred method for introducing foreign DNA into a cell is electroporation.

2. Homologous Recombination

The method of homologous recombination targets the Akt2 gene for disruption by introducing an Akt2 gene targeting vector into a cell containing an Akt2 gene. The ability of the vector to target the Akt2 gene for disruption stems from using a nucleotide sequence in the vector that is homologous, i.e., related, to the Akt2 gene. This homology region facilitates hybridization between the vector and the endogenous sequence of the Akt2 gene. Upon hybridization, the probability of a crossover event between the targeting vector and genomic sequences greatly increases. This crossover event results in the integration of the vector sequence into the Akt2 gene locus and the functional disruption of the Akt2 gene.

General principles regarding the construction of vectors used for targeting are reviewed in Bradley et al. (Biotechnol. 10: 534, 1992). Two different types of vector can be used to insert DNA by homologous recombination: an insertion vector or a replacement vector. An insertion vector is circular DNA which contains a region of Akt2 gene homology with a double stranded break. Following hybridization between the homology region and the endogenous Akt2 gene, a single crossover event at the double stranded break results in the insertion of the entire vector sequence into the endogenous gene at the site of crossover.

The more preferred vector to create the genetically modified non-human mammals and animals cells of the invention by homologous recombination is a replacement vector, which is colinear rather than circular. Replacement vector integration into the Akt2 gene requires a double crossover event, i.e. crossing over at two sites of hybridization between the targeting vector and the Akt2 gene. This double crossover event results in the integration of a vector sequence that is sandwiched between the two sites of crossover into the Akt2 gene and the deletion of the corresponding endogenous Akt2 gene sequence that originally spanned between the two sites of crossover (see, e.g., Thomas and Capecchi et al., Cell 51: 503-12, 1987; Mansour et al., Nature 336: 348-52, 1988; Mansour et al., Proc. Natl. Acad. Sci. USA 87: 7688-7692, 1990; and Mansour, GATA 7: 219-227, 1990).

A region of homology in a targeting vector used to create the genetically modified non-human mammals and animal cells of the invention is generally at least 100 nucleotides in length. Most preferably, the homology region is at least 1-5 kilobases (kb) in length. Although there is no demonstrated minimum length or minimum degree of relatedness required for a homology region, targeting efficiency for homologous recombination generally corresponds with the length and the degree of relatedness between the targeting vector and the Akt2 gene locus. In the case where a replacement vector is used, and a portion of the endogenous Akt2 gene is deleted upon homologous recombination, an additional consideration is the size of the deleted portion of the endogenous Akt2 gene. If this portion of the endogenous Akt2 gene is greater than 1 kb in length, then a targeting cassette with regions of homology that are longer than 1 kb is recommended to enhance the efficiency of recombination. Further guidance regarding the selection and use of sequences effective for homologous recombination, based on the present description, is described in the literature (see, e.g., Deng and Capecchi, Mol. Cell. Biol. 12: 3365-3371, 1992; Bollag et al., Annu. Rev. Genet. 23: 199-225,1989; and Waldman and Liskay, Mol. Cell. Biol. 8: 5350-5357, 1988).

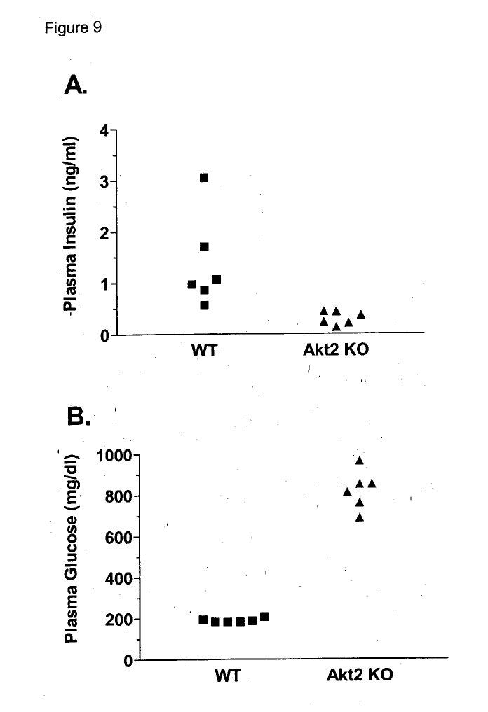

As those skilled in the art will recognize based upon the present invention, a wide variety of cloning vectors may be used as vector backbones in the construction of the Akt2 gene targeting vectors of the present invention, including pBluescript-related plasmids (e.g., Bluescript KS+11), pQE70, pQE60, pQE-9, pBS, pD10, phagescript, phiX174, pBK Phagemid, pNH8A, pNH16a, pNH18Z, pNH46A, ptrc99a, pKK223-3, pKK233-3, pDR540, and pRIT5 PWLNEO, pSV2CAT, pXT1, pSG (Stratagene), pSVK3, PBPV, PMSG, and pSVL, pBR322 and pBR322-based vectors, pMB9, pBR325, pKH47, pBR328, pHC79; phage Charon 28, pKB11, pKSV-10, pK19 related plasmids, pUC plasmids, and the pGEM series of plasmids. These vectors are available from a variety of commercial sources (e.g., Boehringer Mannheim Biochemicals, Indianapolis, Ind.; Qiagen, Valencia, Calif.; Stratagene, La Jolla, Calif.; Promega, Madison, Wis.; and New England Biolabs, Beverly, Mass.). However, any other vectors, e.g. plasmids, viruses, or parts thereof, may be used as long as they are replicable and viable in the desired host. The vector may also comprise sequences which enable it to replicate in the host whose genome is to be modified. The use of such a vector can expand the interaction period during which recombination can occur, increasing the efficiency of targeting (see Molecular Biology, ed. Ausubel et al, Unit 9.16, FIG. 9.16.1).

The specific host employed for propagating the targeting vectors of the present invention is not critical. Examples include E. coli K12 RR1 (Bolivar et al., Gene 2: 95, 1977), E. coli K12 HB101 (ATCC No. 33694), E. coli MM21 (ATCC No. 336780), E. coli DH1 (ATCC No. 33849), E. coli strain DH5α, and E. coli STBL2. Alternatively, hosts such as C. cerevisiae or B. subtilis can be used. The above-mentioned hosts are available commercially (e.g., Stratagene, La Jolla, Calif.; and Life Technologies, Rockville, Md.).

To create the targeting vector, an Akt2 gene targeting construct is added to an above-described vector backbone. The Akt2 gene targeting constructs of the invention have at least one Akt2 gene homology region. To make the Akt2 gene homology regions, an Akt2 genomic or cDNA sequence is used as a basis for producing PCR primers. These primers are used to amplify the desired region of the Akt2 sequence by high fidelity PCR amplification (Mattila et al., Nucleic Acids Res. 19: 4967, 1991; Eckert and Kunkel 1: 17, 1991; and U.S. Pat. No. 4,683,202). The genomic sequence is obtained from a genomic clone library or from a preparation of genomic DNA, preferably from the animal species that is to be targeted for Akt2 gene disruption. An Akt2 cDNA sequence can be used in making an Akt2 targeting vector (e.g., Genbank U22445 (murine), Genbank M95936 (human), Genbank D30041 (rat), Genbank AF181260 (hen), BG410200 (Xenopus)).

Preferably, the targeting constructs of the invention also include an exogenous nucleotide sequence encoding a positive marker protein. The stable expression of a positive marker after vector integration confers an identifiable characteristic on the cell, ideally, without compromising cell viability. Therefore, in the case of a replacement vector, the marker gene is positioned between two flanking homology-regions so that it integrates into the Akt2 gene following the double crossover event in a manner such that the marker gene is positioned for expression after integration.

It is preferred that the positive marker protein is a selectable protein; the stable expression of such a protein in a cell confers a selectable phenotypic characteristic, i.e., the characteristic enhances the survival of the cell under otherwise lethal conditions. Thus, by imposing the selectable condition, one can isolate cells that stably express the positive selectable marker-encoding vector sequence from other cells that have not successfully integrated the vector sequence on the basis of viability. Examples of positive selectable marker proteins (and their agents of selection) include neo (G418 or kanomycin), hyg (hygromycin), hisD (histidinol), gpt (xanthine), ble (bleomycin), and hprt (hypoxanthine) (see, e.g., Capecchi and Thomas, U.S. Pat. No. 5,464,764, and Capecchi, Science 244: 1288-92, 1989). Other positive markers that may also be used as an alternative to a selectable marker include reporter proteins such as β-galactosidase, firefly luciferase, or GFP (see, e.g., Current Protocols in Cytometry, Unit 9.5, and Current Protocols in Molecular Biology, Unit 9.6, John Wiley & Sons, New York, N.Y., 2000).

The above-described positive selection step does not distinguish between cells that have integrated the vector by targeted homologous recombination at the Akt2 gene locus versus random, non-homologous integration of vector sequence into any chromosomal position. Therefore, when using a replacement vector for homologous recombination to make the genetically modified non-human mammals and animal cells of the invention, it is also preferred to include a nucleotide sequence encoding a negative selectable marker protein. Expression of a negative selectable marker causes a cell expressing the marker to lose viability when exposed to a certain agent (i.e., the marker protein becomes lethal to the cell under certain selectable conditions). Examples of negative selectable markers (and their agents of lethality) include herpes simplex virus thymidine kinase (gancyclovir or 1,2-deoxy-2-fluoro-α-d-arabinofuransyl-5-iodouracil), Hprt (6-thioguanine or 6-thioxanthine), and diphtheria toxin, ricin toxin, and cytosine deaminase (5-fluorocytosine).

The nucleotide sequence encoding the negative selectable marker is positioned outside of the two homology regions of the replacement vector. Given this positioning, cells will only integrate and stably express the negative selectable marker if integration occurs by random, non-homologous recombination; homologous recombination between the Akt2 gene and the two regions of homology in the targeting construct excludes the sequence encoding the negative selectable marker from integration. Thus, by imposing the negative condition, cells that have integrated the targeting vector by random, non-homologous recombination lose viability.

The above-described combination of positive and negative selectable markers is preferred in a targeting construct used to make the genetically modified non-human mammals and animal cells of the invention because a series of positive and negative selection steps can be designed to more efficiently select only those cells that have undergone vector integration by homologous recombination, and, therefore, have a potentially disrupted Akt2 gene. Further examples of positive-negative selection schemes, selectable markers, and targeting constructs are described, for example, in U.S. Pat. No. 5,464,764, WO 94/06908, U.S. Pat. No. 5,859,312, and Valancius and Smithies, Mol. Cell. Biol. 11: 1402, 1991.

In order for a marker protein to be stably expressed upon vector integration, the targeting vector may be designed so that the marker coding sequence is operably linked to the endogenous Akt2 gene promoter upon vector integration. Expression of the marker is then driven by the Akt2 gene promoter in cells that normally express the Akt2 gene. Alternatively, each marker in the targeting construct of the vector may contain its own promoter that drives expression independent of the Akt2 gene promoter. This latter scheme has the advantage of allowing for expression of markers in cells that do not typically express the Akt2 gene (Smith and Berg, Cold Spring Harbor Symp. Quant. Biol. 49: 171, 1984; Sedivy and Sharp, Proc. Natl. Acad. Sci. (USA) 86: 227, 1989; Thomas and Capecchi, Cell 51: 503,1987).

Exogenous promoters that can be used to drive marker gene expression include cell-specific or stage-specific promoters, constitutive promoters, and inducible or regulatable promoters. Non-limiting examples of these promoters include the herpes simplex thymidine kinase promoter, cytomegalovirus (CMV) promoter/enhancer, SV40 promoters, PGK promoter, PMC1-neo, metallothionein promoter, adenovirus late promoter, vaccinia virus 7.5K promoter, avian beta globin promoter, histone promoters (e.g., mouse histone H3-614), beta actin promoter, neuron-specific enolase, muscle actin promoter, and the cauliflower mosaic virus 35S promoter (see generally, Sambrook et al., Molecular Cloning, Vols. I-III, Cold Spring Harbor Laboratory Press, Cold Spring Harbor, N.Y., 1989, and Current Protocols in Molecular Biology, John Wiley & Sons, New York, N.Y., 2000; Stratagene, La Jolla, Calif.).

To confirm whether cells have integrated the vector sequence into the targeted Akt2 gene locus while making the genetically modified non-human mammals and animal cells of the invention, primers or genomic probes that are specific for the desired vector integration event can be used in combination with PCR or Southern blot analysis to identify the presence of the desired vector integration into the Akt2 gene locus (Erlich et al., Science 252: 1643-51, 1991; Zimmer and Gruss, Nature 338: 150, 1989; Mouellic et al., Proc. Natl. Acad. Sci. (USA) 87: 4712, 1990; and Shesely et al., Proc. Natl. Acad. Sci. (USA) 88: 4294, 1991).

3. Gene Trapping

Another method available for inserting a foreign nucleic acid sequence into the Akt2 gene locus to disrupt the Akt2 gene, based on the present description, is gene trapping. This method takes advantage of the cellular machinery present in all mammalian cells that splices exons into mRNA to insert a gene trap vector coding sequence into a gene in a random fashion. Once inserted, the gene trap vector creates a mutation that may disrupt the trapped Akt2 gene. In contrast to homologous recombination, this system for mutagenesis creates largely random mutations. Thus, to obtain a genetically-modified cell that contains a disrupted Akt2 gene, cells containing this particular mutation must be identified and selected from a pool of cells that contain random mutations in a variety of genes.

Gene trapping systems and vectors have been described for use in genetically modifying murine cells and other cell types (see, e.g., Allen et al., Nature 333: 852-55, 1988; Bellen et al., Genes Dev. 3: 1288-1300, 1989; Bier et al., Genes Dev. 3: 1273-1287, 1989; Bonnerot et al., J. Virol. 66: 4982-91,1992; Brenner et al., Proc. Nat. Acad. Sci. USA 86: 5517-21, 1989; Chang et al., Virology 193: 737-47, 1993; Friedrich and Soriano, Methods Enzymol. 225: 681-701, 1993; Friedrich and Soriano, Genes Dev. 5: 1513-23, 1991; Goff, Methods Enzymol. 152: 469-81, 1987; Gossler et al., Science 244: 463-65, 1989; Hope, Develop. 113: 399-408, 1991; Kerr et al., Cold Spring Harb. Symp. Quant. Biol. 2: 767-776, 1989; Reddy et al., J. Virol. 65: 1507-1515, 1991; Reddy et al., Proc. Natl. Acad. Sci. U.S.A. 89: 6721-25, 1992; Skarnes et al., Genes Dev. 6: 903-918, 1992; von Melchner and Ruley, J. Virol. 63: 3227-3233, 1989; and Yoshida et al., Transgen. Res. 4: 277-87, 1995).

Promoter trap, or 5′, vectors contain, in 5′ to 3′ order, a splice acceptor sequence followed by an exon, which is typically characterized by a translation initiation codon and open reading frame and/or an internal ribosome entry site. In general, these promoter trap vectors do not contain promoters or operably linked splice donor sequences. Consequently, after integration into the cellular genome of the host cell, the promoter trap vector sequence intercepts the normal splicing of the upstream gene and acts as a terminal exon. Expression of the vector coding sequence is dependent upon the vector integrating into an intron of the disrupted gene in the proper reading frame. In such a case, the cellular splicing machinery splices exons from the trapped gene upstream of the vector coding sequence (Zambrowicz et al., WO 99/50426 and U.S. Pat. No. 6,080,576).

An alternative method for producing an effect similar to the above-described promoter trap vector is a vector that incorporates a nested set of stop codons present in, or otherwise engineered into, the region between the splice acceptor of the promoter trap vector and the translation initiation codon or polyadenylation sequence. The coding sequence can also be engineered to contain an independent ribosome entry site (IRES) so that the coding sequence will be expressed in a manner largely independent of the site of integration within the host cell genome. Typically, but not necessarily, an IRES is used in conjunction with a nested set of stop codons.

Another type of gene trapping scheme uses a 3′ gene trap vector. This type of vector contains, in operative combination, a promoter region, which mediates expression of an adjoining coding sequence, the coding sequence, and a splice donor sequence that defines the 3′ end of the coding sequence exon. After integration into a host cell genome, the transcript expressed by the vector promoter is spliced to a splice acceptor sequence from the trapped gene that is located downstream of the integrated gene trap vector sequence. Thus, the integration of the vector results in the expression of a fusion transcript comprising the coding sequence of the 3′ gene trap cassette and any downstream cellular exons, including the terminal exon and its polyadenylation signal. When such vectors integrate into a gene, the cellular splicing machinery splices the vector coding sequence upstream of the 3′ exons of the trapped gene. One advantage of such vectors is that the expression of the 3′ gene trap vectors is driven by a promoter within the gene trap cassette and does not require integration into a gene that is normally expressed in the host cell (Zambrowicz et al., WO 99/50426 and U.S. Pat. No. 6,080,576). Examples of transcriptional promoters and enhancers that may be incorporated into the 3′ gene trap vector include those discussed above with respect to targeting vectors.

The viral vector backbone used as the structural component for the promoter or 3′ gene trap vector may be selected from a wide range of vectors that can be inserted into the genome of a target cell. Suitable backbone vectors include, but are not limited to, herpes simplex virus vectors, adenovirus vectors, adeno-associated virus vectors, retroviral vectors, lentiviral vectors, pseudorabies virus, alpha-herpes virus vectors, and the like. A thorough review of viral vectors, in particular, viral vectors suitable for modifying nonreplicating cells and how to use such vectors in conjunction with the expression of an exogenous polynucleotide sequence, can be found in Viral Vectors: Gene Therapy and Neuroscience Applications, Eds. Caplitt and Loewy, Academic Press, San Diego, 1995.

Preferably, retroviral vectors are used for gene trapping. These vectors can be used in conjunction with retroviral packaging cell lines such as those described in U.S. Pat. No. 5,449,614. Where non-murine mammalian cells are used as target cells for genetic modification, amphotropic or pantropic packaging cell lines can be used to package suitable vectors (Ory et al., Proc. Natl. Acad. Sci., USA 93: 11400-11406, 1996). Representative retroviral vectors that can be adapted to create the presently described 3′ gene trap vectors are described, for example, in U.S. Pat. No. 5,521,076.

The gene trapping vectors may contain one or more of the positive marker genes discussed above with respect to targeting vectors used for homologous recombination. Similar to their use in targeting vectors, these positive markers are used in gene trapping vectors to identify and select cells that have integrated the vector into the cell genome. The marker gene may be engineered to contain an independent ribosome entry site (IRES) so that the marker will be expressed in a manner largely independent of the location in which the vector has integrated into the target cell genome.

Given that gene trap vectors will integrate into the genome of infected host cells in a fairly random manner, a genetically-modified cell having a disrupted Akt2 gene must be identified from a population of cells that have undergone random vector integration. Preferably, the genetic modifications in the population of cells are of sufficient randomness and frequency such that the population represents mutations in essentially every gene found in the cell's genome, making it likely that a cell with a disrupted Akt2 gene will be identified from the population (see Zambrowicz et al., WO 99/50426; Sands et al., WO 98/14614 and U.S. Pat. No. 6,080,576).

Individual mutant cell lines containing a disrupted Akt2,gene are identified in a population of mutated cells using, for example, reverse transcription and polymerase chain reaction (PCR) to identify a mutation in an Akt2 gene sequence. This process can be streamlined by pooling clones. For example, to find an individual clone containing a disrupted Akt2 gene, RT-PCR is performed using one primer anchored in the gene trap vector and the other primer located in the Akt2 gene sequence. A positive RT-PCR result indicates that the vector sequence is encoded in the Akt2 gene transcript, indicating that the Akt2 gene has been disrupted by a gene trap integration event (see, e.g., Sands et al., WO 98/14614, U.S. Pat. No. 6,080,576).

4. Temporal, Spatial, and Inducible Akt2 Gene Disruptions

In certain embodiments of the present invention, a functional disruption of the endogenous Akt2 gene occurs at specific developmental or cell cycle stages (temporal disruption) or in specific cell types (spatial disruption). In other embodiments, the Akt2 gene disruption is inducible when certain conditions are present. A recombinase excision system, such as a Cre-Lox system, may be used to activate or inactivate the Akt2 gene at a specific developmental stage, in a particular tissue or cell type, or under particular environmental conditions. Generally, methods utilizing Cre-Lox technology are carried out as described by Torres and Kuhn, Laboratory Protocols for Conditional Gene Targeting, Oxford University Press, 1997. Methodology similar to that described for the Cre-Lox system can also be employed utilizing the FLP-FRT system. Further guidance regarding the use of recombinase excision systems for conditionally disrupting genes by homologous recombination or viral insertion is provided, for example, in U.S. Pat. No. 5,626,159, U.S. Pat. No. 5,527,695, U.S. Pat. No. 5,434,066, WO 98/29533, U.S. Pat. No. 6,228,639, Orban et al., Proc. Nat. Acad. Sci. USA 89: 6861-65, 1992; O'Gorman et al., Science 251: 1351-55, 1991; Sauer et al., Nucleic Acids Research 17: 147-61, 1989; Barinaga, Science 265: 26-28, 1994; and Akagi et al., Nucleic Acids Res. 25: 1766-73, 1997. More than one recombinase system can be used to genetically modify a non-human mammal or animal cell of the present invention.

When using homologous recombination to disrupt the Akt2 gene in a temporal, spatial, or inducible fashion, using a recombinase system such as the Cre-Lox system, a portion of the Akt2 gene coding region is replaced by a targeting construct comprising the Akt2 gene coding region flanked by loxP sites. Non-human mammals and animal cells carrying this genetic modification contain a functional, loxp-flanked Akt2 gene. The temporal, spatial, or inducible aspect of the Akt2 gene disruption is caused by the expression pattern of an additional transgene, a Cre recombinase transgene, that is expressed in the non-human mammal or animal cell under the control of the desired spatially-regulated, temporally-regulated, or inducible promoter, respectively. A Cre recombinase targets the loxP sites for recombination. Therefore, when Cre expression is activated, the LoxP sites undergo recombination to excise the sandwiched Akt2 gene coding sequence, resulting in a functional disruption of the Akt2 gene (Rajewski et al., J. Clin. Invest. 98: 600-03, 1996; St.-Onge et al., Nucleic Acids Res. 24: 3875-77, 1996; Agah et al., J. Clin. Invest. 100: 169-79, 1997; Brocard et al., Proc. Natl. Acad. Sci. USA 94: 14559-63, 1997; Feil et al., Proc. Natl. Acad. Sci. USA 93: 10887-90, 1996; and Kuhn et al., Science 269: 1427-29, 1995).

A cell containing both a Cre recombinase transgene and loxP-flanked Akt2 gene can be generated through standard transgenic techniques or, in the case of genetically-modified, non-human mammals, by crossing genetically-modified, non-human mammals wherein one parent contains a loxP flanked Akt2 gene and the other contains a Cre recombinase transgene under the control of the desired promoter. Further guidance regarding the use of recombinase systems and specific promoters to temporally, spatially, or conditionally disrupt the Akt2 gene is found, for example, in Sauer, Meth. Enz. 225: 890-900, 1993, Gu et al., Science 265: 103-06, 1994, Araki et al., J. Biochem. 122: 977-82, 1997, Dymecki, Proc. Natl. Acad. Sci. 93: 6191-96, 1996, and Meyers et al., Nature Genetics 18: 136-41, 1998.

An inducible disruption of the Akt2 gene can also be achieved by using a tetracycline responsive binary system (Gossen and Bujard, Proc. Natl. Acad. Sci. USA 89: 5547-51, 1992). This system involves genetically modifying a cell to introduce a Tet promoter into the endogenous Akt2 gene regulatory element and a transgene expressing a tetracycline-controllable repressor (TetR). In such a cell, the administration of tetracycline activates the TetR which, in turn, inhibits Akt2 gene expression and, therefore, disrupts the Akt2 gene (St.-Onge et al., Nucleic Acids Res. 24: 3875-77, 1996, U.S. Pat. No. 5,922,927).

The above-described systems for temporal, spatial, and inducible disruptions of the Akt2 gene can also be adopted when using gene trapping as the method of genetic modification, for example, as described, in WO 98/29533 and U.S. Pat. No. 6,288,639, for creating the genetically modified non-human mammals and animal cells of the invention.

5. Creating Genetically-Modified, Non-human Mammals and Animal Cells

The above-described methods for genetic modification can be used to disrupt an Akt2 gene in virtually any type of somatic or stem cell derived from an animal to create the genetically modified animal cells of the invention. Genetically-modified animal cells of the invention include, but are not limited to, mammalian cells, including human cells, and avian cells. These cells may be derived from genetically engineering any animal cell line, such as culture-adapted, tumorigenic, or transformed cell lines, or they may be isolated from a genetically-modified, non-human mammal carrying the desired Akt2 genetic modification.

The cells may be heterozygous or homozygous for the disrupted Akt2 gene. To obtain cells that are homozygous for the Akt2 gene disruption (−/−), direct, sequential targeting of both alleles can be performed. This process can be facilitated by recycling a positive selectable marker. According to this scheme the nucleotide sequence encoding the positive selectable marker is removed following the disruption of one allele using the Cre-Lox P system. Thus, the same vector can be used in a subsequent round of targeting to disrupt the second Akt2 gene allele (Abuin and Bradley, Mol. Cell. Biol. 16: 1851-56, 1996; Sedivy et al., T.I.G. 15: 88-90, 1999; Cruz et al., Proc. Natl. Acad. Sci. (USA) 88: 7170-74, 1991; Mortensen et al., Proc. Natl. Acad. Sci. (USA) 88: 7036-40, 1991; te Riele et al., Nature (London) 348: 649-651, 1990).

An alternative strategy for obtaining ES cells that are Akt2−/− is the homogenotization of cells from a population of cells that is heterozygous for the Akt2 gene disruption (Akt2±). The method uses a scheme in which Akt2± targeted clones that express a selectable drug resistance marker are selected against a very high drug concentration; this selection favors cells that express two copies of the sequence encoding the drug resistance marker and are, therefore, homozygous for the Akt2 gene disruption (Mortensen et al., Mol. Cell. Biol. 12: 2391-95, 1992). In addition, genetically-modified animal cells can be obtained from genetically-modified Akt2−/− non-human mammals that are created by mating non-human mammals that are Akt2± in germline cells, as further discussed below.

Following the genetic modification of the desired cell or cell line, the Akt2 gene locus can be confirmed as the site of modification by PCR analysis according to standard PCR or Southern blotting methods known in the art (see, e.g., U.S. Pat. No. 4,683,202; and Erlich et al., Science 252: 1643, 1991). Further verification of the functional disruption of the Akt2 gene may also be made if Akt2 gene messenger RNA (mRNA) levels and/or Akt2 polypeptide levels are reduced in cells that normally express the Akt2 gene. Measures of Akt2 gene mRNA levels may be obtained by using reverse transcriptase mediated polymerase chain reaction (RT-PCR), Northern blot analysis, or in situ hybridization. The quantification of Akt2 polypeptide levels produced by the cells can be made, for example, by standard immunoassay methods known in the art. Such immunoassays include, but are not limited to, competitive and non-competitive assay systems using techniques such as RIAs (radioimmunoassays), ELISAs (enzyme-linked immunosorbent assays), “sandwich” immunoassays, immunoradiometric assays, gel diffusion precipitin reactions, immunodiffusion assays, in situ immunoassays (using, for example, colloidal gold, enzymatic, or radioisotope labels), Western blots, 2-dimensional gel analysis, precipitation reactions, immunofluorescence assays, protein A assays, and immunoelectrophoresis assays.

Preferred genetically-modified animal cells of the invention are embryonic stem (ES) cells and ES-like cells. These cells are derived from the preimplantation embryos and blastocysts of various species, such as mice (Evans et al., Nature 129:154-156, 1981; Martin, Proc. Natl. Acad. Sci., USA, 78: 7634-7638, 1981), pigs and sheep (Notanianni et al., J. Reprod. Fert. Suppl., 43: 255-260, 1991; Campbell et al., Nature 380: 64-68, 1996) and primates, including humans (Thomson et al., U.S. Pat. No. 5,843,780, Thomson et al., Science 282: 1145-1147, 1995; and Thomson et al., Proc. Natl. Acad. Sci. USA 92: 7844-7848, 1995).

These types of cells are pluripotent, that is, under proper conditions, they differentiate into a wide variety of cell types derived from all three embryonic germ layers: ectoderm, mesoderm and endoderm. Depending upon the culture conditions, a sample of ES cells can be cultured indefinitely as stem cells, allowed to differentiate into a wide variety of different cell types within a single sample, or directed to differentiate into a specific cell type, such as macrophage-like cells, neuronal cells, cardiomyocytes, chondrocytes, adipocytes, smooth muscle cells, endothelial cells, skeletal muscle cells, keratinocytes, and hematopoietic cells, such as eosinophils, mast cells, erythroid progenitor cells, or megakaryocytes. Directed differentiation is accomplished by including specific growth factors or matrix components in the culture conditions, as further described, for example, in Keller et al., Curr. Opin. Cell Biol. 7: 862-69, 1995, Li et al., Curr. Biol. 8: 971,1998, Klug et al., J. Clin. Invest. 98: 216-24, 1996, Lieschke et al., Exp. Hematol. 23: 328-34, 1995, Yamane et al., Blood 90: 3516-23, 1997, and Hirashima et al., Blood 93: 1253-63, 1999.

The particular embryonic stem cell line that is used for genetic modification is not critical; exemplary murine ES cell lines include AB-1 (McMahon and Bradley, Cell 62:1073-85, 1990), E14 (Hooper et al., Nature 326: 292-95, 1987), D3 (Doetschman et al., J. Embryol. Exp. Morph. 87: 27-45, 1985), CCE (Robertson et al, Nature 323: 445-48, 1986), RW4 (Genome Systems, St. Louis, Mo.), and DBA/1lacj (Roach et al., Exp. Cell Res. 221: 520-25, 1995). Genetically-modified murine ES cells may be used to generate genetically-modified mice, according to published procedures (Robertson, 1987, Teratocarcinomas and Embryonic Stem Cells: A Practical Approach, Ed. E. J. Robertson, Oxford: IRL Press, pp. 71-112, 1987; Zjilstra et al., Nature 342: 435-438, 1989; and Schwartzberg et al., Science 246: 799-803, 1989).

Following confirmation that the ES cells contain the desired functional disruption of the Akt2 gene, these ES cells are then injected into suitable blastocyst hosts for generation of chimeric mice according to methods known in the art (Capecchi, Trends Genet. 5: 70, 1989). The particular mouse blastocysts employed in the present invention are not critical. Examples of such blastocysts include those derived from C57BL6 mice, C57BL6 Albino mice, Swiss outbred mice, CFLP mice, and MFI mice. Alternatively ES cells may be sandwiched between tetraploid embryos in aggregation wells (Nagy et al., Proc. Natl. Acad. Sci. USA 90: 8424-8428, 1993).

The blastocysts or embryos containing the genetically-modified ES cells are then implanted in pseudopregnant female mice and allowed to develop in utero (Hogan et al., Manipulating the Mouse Embryo: A Laboratory Manual, Cold Spring Harbor Laboratory press, Cold Spring Harbor, N.Y. 1988; and Teratocarcinomas and Embryonic Stem Cells: A Practical Approach, E. J. Robertson, ed., IRL Press, Washington, D.C., 1987). The offspring born to the foster mothers may be screened to identify those that are chimeric for the Akt2 gene disruption. Generally, such offspring contain some cells that are derived from the genetically-modified donor ES cell as well as other cells derived from the original blastocyst. In such circumstances, offspring may be screened initially for mosaic coat color, where a coat color selection strategy has been employed, to distinguish cells derived from the donor ES cell from the other cells of the blastocyst. Alternatively, DNA from tail tissue of the offspring can be used to identify mice containing the genetically-modified cells.

The mating of chimeric mice that contain the Akt2 gene disruption in germ line cells produces progeny that possess the Akt2 gene disruption in all germ line cells and somatic cells. Mice that are heterozygous for the Akt2 gene disruption can then be crossed to produce homozygotes (see, e.g., U.S. Pat. No. 5,557,032, and U.S. Pat. No. 5,532,158).

An alternative to the above-described ES cell technology for transferring a genetic modification from a cell to a whole animal is to use nuclear transfer. This method can be employed to make other genetically-modified, non-human mammals besides mice, for example, sheep (McCreath et al., Nature 29: 1066-69, 2000; Campbell et al., Nature 389: 64-66, 1996; and Schnieke et al., Science 278: 2130-33, 1997) and calves (Cibelli et al., Science 280: 1256-58, 1998). Briefly, somatic cells (e.g., fibroblasts) or pluripotent stem cells (e.g., ES-like cells) are selected as nuclear donors and are genetically-modified to contain a functional disruption of the Akt2 gene. When inserting a DNA vector into a somatic cell to mutate the Akt2 gene, it is preferred that a promoterless marker be used in the vector such that vector integration into the Akt2 gene results in expression of the marker under the control of the Akt2 gene promoter (Sedivy and Dutriaux, T.I.G. 15: 88-90, 1999; McCreath et al., Nature 29: 1066-69, 2000). Nuclei from donor cells which have the appropriate Akt2 gene disruption are then transferred to fertilized or parthenogenetic oocytes that are enucleated (Campbell et al., Nature 380: 64, 1996; Wilmut et al., Nature 385: 810, 1997). Embryos are reconstructed, cultured to develop into the morula/blastocyst stage, and transferred into foster mothers for full term in utero development.

The present invention also encompasses the progeny of the genetically-modified, non-human mammals and genetically-modified animal cells. While the progeny are heterozygous or homozygous for the genetic modification that disrupts the Akt2 gene, they may not be genetically identical to the parent non-human mammals and animal cells due to mutations or environmental influences, besides that of the original genetic disruption of the Akt2 gene, that may occur in succeeding generations.

The cells from a non-human genetically modified animal can be isolated from tissue or organs using techniques known to those of skill in the art. In one embodiment, the genetically modified cells of the invention are immortalized. In accordance with this embodiment, cells can be immortalized by genetically engineering the telomerase gene, an oncogene, e.g., mos or v-src, or an apoptosis-inhibiting gene, e.g., bcl-2, into the cells. Alternatively, cells can be immortalized by fusion with a hybridization partner utilizing techniques known to one of skill in the art.

6. “Humanized” Non-human Mammals and Animal Cells

The genetically-modified non-human mammals and animal cells (non-human) of the invention containing a disrupted endogenous Akt2 gene can be further modified to express the human Akt2 sequence (referred to herein as “humanized”). A preferred method for humanizing cells involves replacing the endogenous Akt2 sequence with nucleic acid sequence encoding the human Akt2 sequence (Jakobsson et al., Proc. Natl. Acad. Sci. USA 96: 7220-25, 1999) by homologous recombination. The vectors are similar to those traditionally used as targeting vectors with respect to the 5′ and 3′ homology arms and positive/negative selection schemes. However, the vectors also include sequence that, after recombination, either substitutes the human Akt2 coding sequence for the endogenous sequence, or effects base pair changes, exon substitutions, or codon substitutions that modify the endogenous sequence to encode the human Akt2. Once homologous recombinants have been identified, it is possible to excise any selection-based sequences (e.g., neo) by using Cre or Flp-mediated site directed recombination (Dymecki, Proc. Natl. Acad. Sci. 93: 6191-96, 1996).

When substituting the human Akt2 sequence for the endogenous sequence, it is preferred that these changes are introduced directly downstream of the endogenous translation start site. This positioning preserves the endogenous temporal and spatial expression patterns of the Akt2 gene. The human sequence can be the full length human cDNA sequence with a polyA tail attached at the 3′ end for proper processing or the whole genomic sequence (Shiao et al., Transgenic Res. 8: 295-302, 1999). Further guidance regarding these methods of genetically modifying cells and non-human mammals to replace expression of an endogenous gene with its human counterpart is found, for example, in Sullivan et al., J. Biol. Chem. 272: 17972-80, 1997, Reaume et al., J. Biol. Chem. 271: 23380-88,1996, and Scott et al., U.S. Pat. No. 5,777,194).

Another method for creating such “humanized” organisms is a two step process involving the disruption of the endogenous gene followed by the introduction of a transgene encoding the human sequence by pronuclear microinjection into the knock-out embryos.

7. Uses for the Genetically-Modified Non-Human Mammals and Animal Cells

The genetically-modified mice homozygous (−/−) for the Akt2 disruption demonstrate elevated plasma glucose and plasma insulin levels (as shown in the examples below), and also demonstrate impaired glucose tolerance (Cho et al., Science 292: 1728-31, 2001). Therefore the Akt2−/− mice can be used as diabetic models to screen for anti-diabetic agents. Test agents are administered to the mice via any standard route and screening is conducted to identify agents that cause an improvement in a diabetic phenotype. Preferably, test agents are screened to identify agents that cause a decrease in plasma glucose and/or a decrease in plasma insulin levels. Agents can also be tested to identify those that cause an improved glucose tolerance following an oral, intraperitoneal, or intravenous glucose tolerance test.

Given that the Akt2−/− mice also develop elevated levels of Tau phosphorylation in the brain (see the Examples herein), these mice are also useful as models of Alzheimer's disease and can be used to screen for agents that treat Alzheimer's disease. Following administration of a test agent though a standard route, brains from mice can be assessed to identify those agents that improve a symptom of Alzheimer's disease. Preferably, agents are identified that reduce tau phosphorylation in the brain using standard methods, as further described in the Examples herein. Briefly, agents can be administered subcutaneously, intraperitoneally, or by oral gavage. Following a delay (e.g., 30 mins to 3 hours), brains are excised from sacrificed animals, homogenized in buffer, and the tau phosphorylation status is assessed with a solution sandwich immunoassay using, for example, the phosphoepitope-selective antibody AT-8 for detection. Alternatively, such screens can be conducted in a cell or tissue-based assay using a primary neuron preparation, a cultured neuronal cell line, or brain slices.

Akt2 function and therapeutic relevance can be further elucidated by investigating the phenotype of Akt2−/− non-human mammals and animals cells of the invention. For example, the genetically-modified Akt2−/− non-human mammals and animal cells can be used to determine whether the Akt2 plays a role in causing or preventing symptoms or phenotypes to develop in certain models of disease, e.g., diabetes/insulin resistance, Alzheimer's Disease, and cancer. If a symptom or phenotype is different in an Akt2−/− non-human mammal or animal cell as compared to a wild type (Akt2+/+) or Akt2± non-human mammal or animal cell, then the Akt2 polypeptide plays a role in regulating functions associated with the symptom or phenotype. Examples of testing that can be used to assess Akt2 function include comparing Akt2−/− mice to wild type mice in terms of glucose/insulin metabolism (e.g., glucose uptake in isolated tissues, alterations in the activity of glycogen metabolism enzymes, alterations in glycogen levels in liver or muscle, and/or alterations in body composition), changes in the activity or phosphorylation state of components in the insulin signaling pathway, Tau phosphorylation, and susceptibility to tumorigenesis.

Exemplary methods of assessing such parameters are provided in the Examples herein. Tumorigenic susceptibility can be assessed, for example, in mice that are the product of crossing Akt2−/− mice to an oncomouse strain (e.g., p53−/−, PTEN−/−, or TRAMP). Skin carcinogenesis can be assessed, for example, by measuring papillomas in mice subject to a single dimethylbenz[a]anthracene (DMBA) treatment followed by TPA-acetone treatments twice a week for approximately 15 weeks. In addition, cells derived from Akt2−/− mice, for example, mouse embryo fibroblasts, can be tested for their susceptibility to cellular oncogenic transformation by transfecting the cells with Ras and myc, or with Ras and dominant negative p53.

In addition, under circumstances in which an agent has been identified as an Akt2 agonist or antagonist (e.g., the agent significantly modifies one or more of the Akt2 polypeptide activities when the agent is administered to an Akt2+/+ or Akt2± non-human mammal or animal cell), the genetically-modified Akt2−/− non-human mammals and animal cells of the invention are useful to characterize any other effects caused by the agent besides those known to result from the (ant)agonism of Akt2 (i.e., the non-human mammals and animal cells can be used as negative controls). For example, if the administration of the agent causes an effect in an Akt2+/+ non-human mammal or animal cell that is not known to be associated with Akt2 polypeptide activity, then one can determine whether the agent exerts this effect solely or primarily through modulation of Akt2 by administering the agent to a corresponding Akt2−/− non-human mammal or animal cell. If this effect is absent, or is significantly reduced, in the Akt2−/− non-human mammal or animal cell, then the effect is mediated, at least in part, by Akt2. However, if the Akt2−/− non-human mammal or animal cell exhibits the effect to a degree comparable to the Akt2+/+ or Akt2± non-human mammal or animal cell, then the effect is mediated by a pathway that does not involve Akt2 signaling.

Furthermore, if an agent is suspected of possibly exerting an effect predominantly via an Akt2 pathway, then the Akt2−/− non-human mammals and animal cells are useful as negative controls to test this hypothesis. If the agent is indeed acting through Akt2, then the Akt2−/− non-human mammals and animal cells, upon administration of the agent, should not demonstrate the same effect observed in the Akt2+/+ non-human mammals or animal cells.

The genetically modified non-human mammals and animal cells of the invention can also be used to identify genes whose expression is differentially regulated in Akt2± or Akt2−/− non-human mammals or animal cells relative to their respective wild-type control. Techniques known to those of skill in the art can be used to identify such genes based upon the present description. For example, DNA arrays can be used to identify genes whose expression is differentially regulated in Akt2± or Akt2−/− mice to compensate for a deficiency in Akt2 expression. DNA arrays are known to those of skill in the art (see, e.g., Aigner et al., Arthritis and Rheumatism 44: 2777-89, 2001; U.S. Pat. No. 5,965,352; Schena et al., Science 270: 467-470, 1995; Schena et al., Proc. Natl. Acad. Sci. USA 93: 10614-10619, 1996; DeRisi et al., Nature Genetics 14: 457-460, 1996; Shalon et al., Genome Res. 6: 639-645, 1996; and Schena et al., Proc. Natl. Acad. Sci. (USA) 93: 10539-11286, 1995; U.S. Pat. No. 5,474,796; U.S. Pat. No. 5,605,662; WO 95/25116; WO 95/35505; Heller et al., Proc. Natl. Acad. Sci. 94: 2150-2155,1997).

A chemical coupling procedure and an ink jet device may be used to synthesize array elements on the surface of a substrate. An array analogous to a dot or slot blot may also be used to arrange and link elements to the surface of a substrate using thermal, UV, chemical, or mechanical bonding procedures. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each probe which hybridizes to an element on the microarray may be assessed through analysis of the scanned images.

Full-length cDNAs, expressed sequence tags (ESTs), or fragments thereof may comprise the elements of a microarray. Fragments suitable for hybridization may be selected using software well known in the art such as LASERGENE software (DNASTAR). Full-length cDNAs, ESTs, or fragments thereof corresponding to one of the nucleotide sequences of the present invention, or selected at random from a cDNA library relevant to the present invention, are arranged on an appropriate substrate, e.g., a glass slide. The cDNA is fixed to the slide using, e.g., ultra-violet cross-linking followed by thermal and chemical treatments and subsequent drying (see, e.g., Schena et al. (1995); Shalon et al. (1996)). Fluorescent probes are prepared and used for hybridization to the elements on the substrate. The substrate is analyzed by procedures well known in the art, for example, by scanning and analyzing images of a microarray.

In addition, the genetically modified non-human mammals and animal cells of the invention can also be used to identify proteins whose expression profile or postranslational modification is altered in Akt2± or Akt2−/− non-human mammals or animal cells relative to their respective wild-type control. Techniques known to those of skill in the art can be used to identify such proteins based upon the present description. For example, proteomic assays can be used to identify proteins whose expression profile or postranslational modification is altered in Akt2± or Akt2−/− mice to compensate for a deficiency in Akt2 expression. Proteomic assays are known to those of skill in the art (see, e.g., Conrads et al., Biochem. Biophys. Res. Commun. 290: 896-890, 2002; Dongre et al., Bioploymers 60: 206-211, 2001; Van Eyk Curr Opin Mol Ther 3: 546-553, 2001; Cole et al., Electrophoresis 21: 1772-1781, 2000; Araki et al., Electrophoresis 21: 180-1889, 2000).

### EXAMPLES

A. Targeting Vector Construction

A 1.0 kb genomic fragment was used to hybridize a DBA/1 lacJ genomic lambda phage library (Stratagene, La Jolla, Calif.). This genomic fragment contained base pairs 1550-1693 of the murine Akt2 cDNA sequence (Genbank AFU22445). Two overlapping Akt2 genomic clones were isolated and subcloned into the Not I site of pBluescript SK+ (Stratagene).

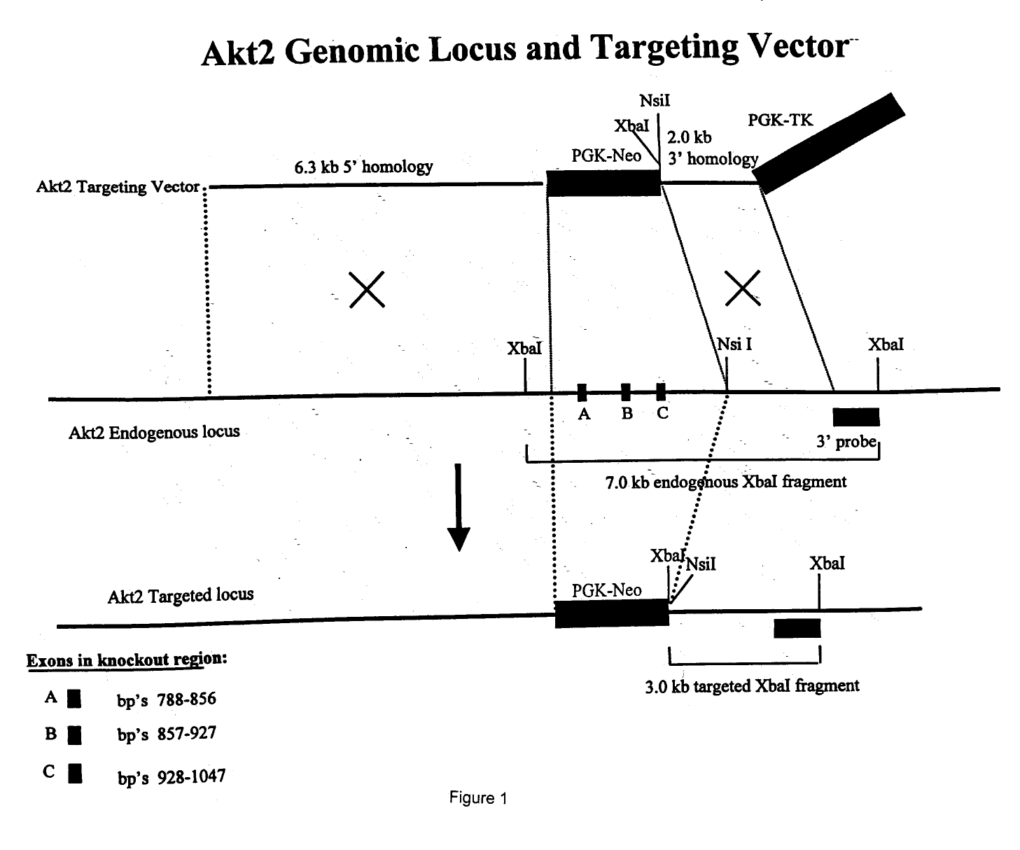

A 3.0 kb Nsi I/Xbal fragment of a genomic clone was isolated and cloned into the Nsi l/Xba I sites of a Litmus 28 cloning vector (New England Biolabs, Beverly, Mass.). This fragment was re-isolated as a 2.0 kb Bgl II/BamHI fragment and cloned into the BamHI site of a pJNS2-Frt targeting vector backbone (Dombrowicz et al., Cell 75: 969-76, 1993) to serve as the 3′ homology arm of the targeting vector. The 5′ homology arm was isolated from an Akt2 genomic clone as a 6.3 kb Not I/Spe I fragment and subcloned into the Not I/Spe I sites of pBluescript SK+. This 6.3 kb fragment was then re-isolated as a Not I/Xho I fragment and subcloned into the Not I/Xho I cloning sites of the pJNS2-Frt targeting vector (which already contained the 3′ homology arm) to serve as the 5′ homology arm and complete the targeting vector. The targeting vector was designed to replace 3.4 kb of the Akt2 genomic locus with the PGK-neomycin cassette (FIG. 1); the deleted 3.4 kb genomic fragment contains 3 exons encoding base pairs 788-1047 of the Akt2 cDNA sequence.

B. ES Cell Screening

The Akt2 targeting vector was linearized with NotI and electroporated into DBA/1 LacJ ES cells (Roach et al., Exp. Cell. Res. 221: 520-25, 1995). Pluripotent ES cells were maintained in culture on a mitomycin C treated primary embryonic fibroblast (PEF) feeder layer in stem cell medium (SCML) which consisted of knockout DMEM (Invitrogen Life Technologies, Inc., (ILTI) Gaithersburg, Md., #10829-018) supplemented with 15% ES cell qualified fetal calf serum (ILTI, #10439-024), 0.1 mM 2-mercaptoethanol (Sigma, St. Louis, Mo., #M-7522), 0.2 mM L-glutamine (ILTI, #25030-081), 0.1 mM MEM non-essential amino acids (ILTI, #11140-050), 1000 units/ml recombinant murine leukemia inhibitory factor (Chemicon International Inc., Temecula, Calif., #ESG-1107), and penicillin/streptomycin (ILTI, #15140-122).

Electroporation of 1×10⁷ cells in SCML and 25 μg linearized targeting vector was carried out using a BTX Electro Cell Manipulator 600 (BTX, Inc., San Diego, Calif.) at a voltage of 240 V, a capacitance of 50 μF, and a resistance of 360 Ohms. Positive/negative selection began 24 hours after electroporation in SCML which contained 200 μg/ml G418 (ILTI, #11811-031) and 2 μM gancyclovir (Syntex Laboratories, Palo Alto, Calif.), as previously described (Mansour et al, Nature 236: 348-52, 1988).

Resistant colonies were picked with a micropipette following 8-12 days of selection. Expansion and screening of resistant ES cell colonies was performed as previously described (Mohn, DNA Cloning 4 (ed. Hames), 143-184, Oxford University Press, New York, 1995).

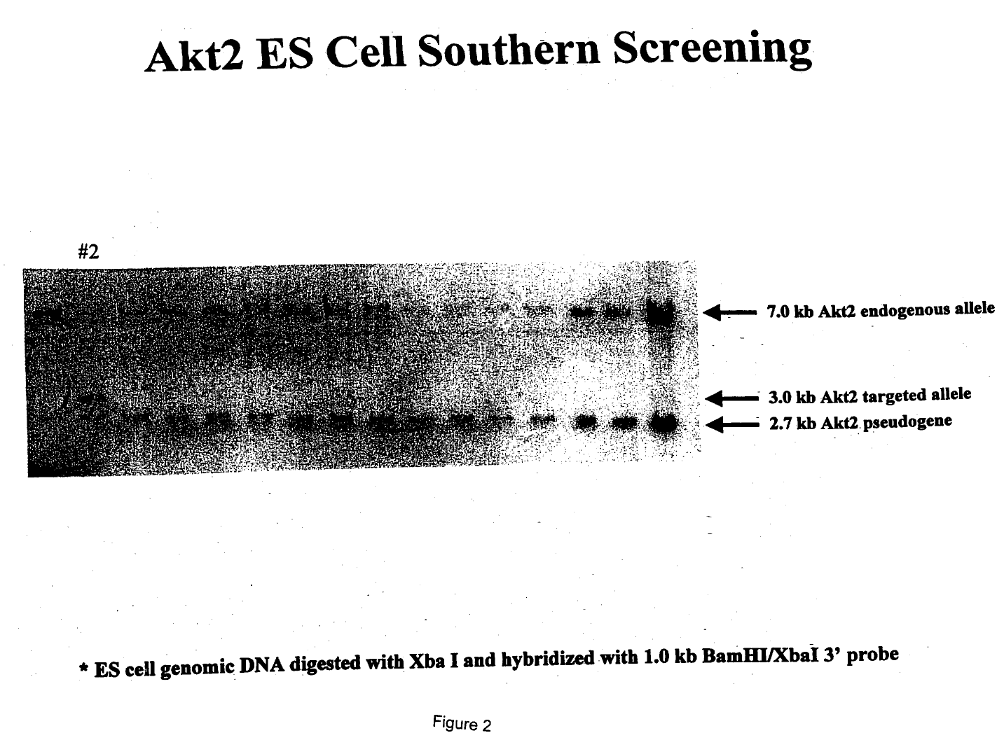

DNA was isolated from ES cell clones which survived G418 and gancyclovir selection. The DNA was digested with Xba I enzyme, electrophoresed on 0.7% agarose gels (BioWhittaker Molecular Applications, Rockland, Me.) and transferred to Hybond N+ nylon membrane (Amersham Pharmacia Biotech, Buckinghamshire, England) for Southern analysis. A 1.0 kb BamHI/Xba I genomic fragment, downstream of the 3′ homology arm was used as a probe to identify homologously recombined ES cells. This 1.0 kb probe recognizes a 7.0 kb endogenous allele Xba I fragment and a 3.0 kb targeted allele due to the introduction of an additional Xba I site in the PGK-neomycin cassette. This probe also recognizes a 2.7 kb Xba I fragment in all ES cells (Akt2 pseudogene). A targeted clone (clone #2) was identified using the 3′ probe (FIG. 2).

C. Knockout Mouse Production

ES cells from clone #2 and another targeted clone (clone #24) were microinjected into blastocyst stage embryos isolated from C57BL/6J females (The Jackson Laboratory, Bar Harbor, Me.). Male chimeras were identified and back-crossed to DBA/1lacJ females (The Jackson Laboratory) to derive germline Akt2 heterozygous (±) offspring.

Normal Mendelian ratios were observed in offspring from these matings. Heterozygous animals were genotyped by PCR for the presence of the neomycin cassette. The primer set consisted of Neo-833F (5′ gcaggatctcctgtcatctcacc 3′) (SEQ ID NO: 1) and Neo-1023R (5′ gatgctcltcgtccagatcatcc 3′) (SEQ ID NO: 2). This oligo set amplified a 190 bp fragment from a targeted Akt2 allele.

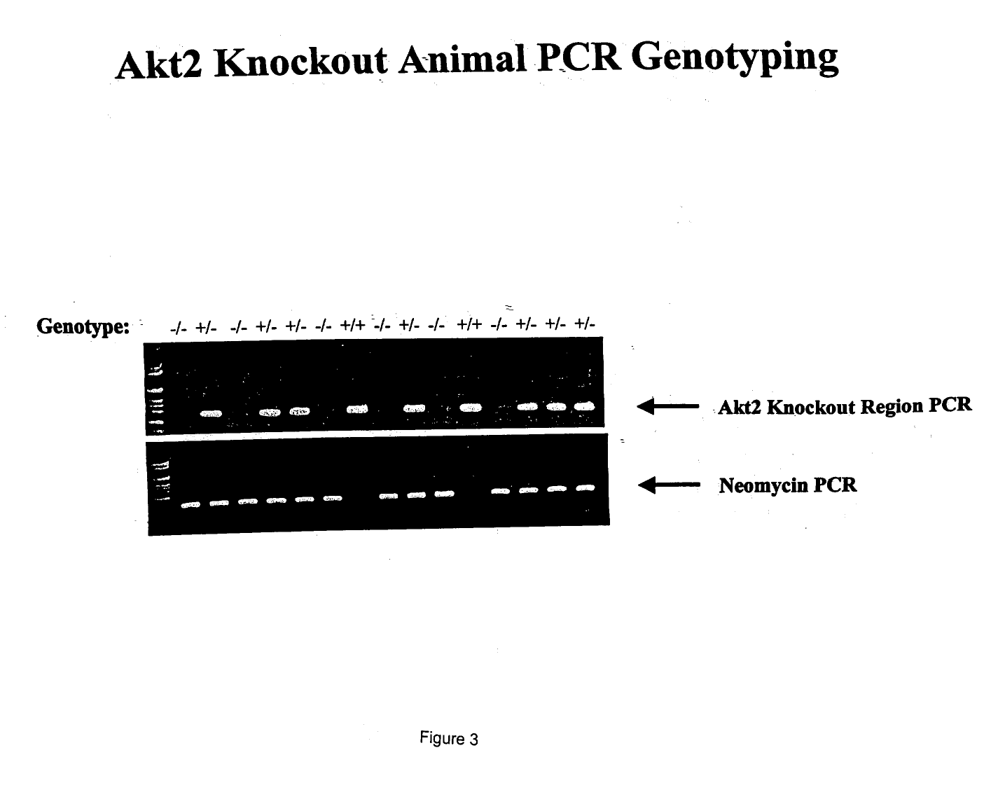

Heterozygous males and females were mated to generate homozygous (−/−) Akt2 knockouts. These offspring were genotyped using a 2 primer set approach, one specific for the neomycin cassette (Neo-833F and Neo-1023R) and the second set specific for the Akt2 knockout region (FIG. 3). The Akt2 specific primer set amplified a 359 bp fragment contained within the 3.4 kb knockout region and consisted of Akt2KO-178F (5′ gaggtagaaacaagagaatcatgg 3′) (SEQ ID NO: 3) and Akt2KO-537R (5′ gttcgcactgctgtatgttgc 3′) (SEQ ID NO: 4).

D. Protein Analysis

Whole brains from wild type (WT) and Akt2 knockout (KO) mice were homogenized in 10 volumes homogenization buffer (50 mM TRIS pH 8.0, 10 mM β-glycerophosphate, 5 mM EGTA, 50 mM NaCl, 10 mM DTT, 1 μM microcystin, 1 mM NaVO₄, 1 mM benzamidine, and 1× Protease Inhibitor Cocktail (Calbiochem, San Diego, Calif.)). The tissues were disrupted using a Polytron and cell debris was cleared by centrifugation at 17,000 ×g (10 min at 4° C.). Supernatants were recovered and protein concentration was determined by Bradford assay. Protein extract (25 ug) was analyzed by SDS-PAGE using a 4-12% NuPage gel in 1× MOPS SDS running buffer (Invitrogen, Carlsbad, Calif., Cat. No. NP0050). Western blot analysis was performed according to manufacturer's instructions using anti-Akt1 (Cat. No. 06-558) and anti-Akt2 (Cat. No. 06-606) antibodies from Upstate Biotechnology (Lake Placid, N.Y.).

F. Phenotypic Characterization of Akt2 Knockout Mice

In summary, the Akt2 KO mouse phenotype demonstrates that Akt2 regulates both growth and glucose metabolism. Akt2 null mice exhibited growth deficiency, lipoatrophy, glucose intolerance, insulin resistance (as evidenced by elevated plasma insulin levels), dyslipidemia, and hyperglycemia. In a substantial portion of males, the condition progressively worsened to a severe form of diabetes that was accompanied by β cell failure and loss. This complex phenotype indicates that Akt2 is a key intermediate in signaling from both insulin and IGF-I receptors and plays a role in the maintenance of adipose tissue and β cell mass. Thus, administering an agent that increases Akt2 activity is useful to treat patients for diabetes and/or growth restriction.

Methods

Oral glucose tolerance test and glucose measurements. Male and female WT and Akt2 KO mice at 7 weeks of age were fasted overnight. A blood sample was collected immediately prior to administration of a glucose load (1 gram of glucose per kg of body weight) by oral gavage using a syringe equipped with a murine oral feeding needle (20 gauge; Popper & Sons, Inc., New Hyde Park, N.Y.). Blood samples were taken at 30, 60, and 120 mins following glucose administration. Blood was immediately diluted into 100 μl of 0.025% heparin in normal saline on ice. Red cells were pelleted by centrifugation at top speed in a Beckman Microfuge 12 for 2 mins. Glucose was determined in the supernatants using the Roche/Hitachi 912 Clinical Chemistry Analyzer (Roche Diagnostics Corp., Indianapolis, Ind.).

Plasma insulin measurements. Insulin was measured in whole plasma, or in samples prepared for glucose measurements (described above), by radioimmunoassay using Linco's Rat Insulin RIA Kit (#RI-13K) or the Sensitive Rat Insulin RIA Kit (#SRI-13K, Linco, St. Charles, Mo.).

Morphometric analysis of the pancreatic islets and adipose tissue. Pancreatic tissues and epididymal fat pads were fixed in formalin, embedded in paraffin, and sections were stained by hematoxylin and eosin. For pancreatic islets in each animal, the area of the whole section of pancreas and of twelve randomly selected islets in that section were measured using Image-Pro Plus® image analysis software (Media Cybernetics, Carlsbad, Calif.). The total number of islets per section was manually counted. The percentage of the surface area occupied by the islets compared to the total area of the pancreas section was calculated using the following formula: mean islet area X total number of islets X 100/total area of the section. The mean number of islets per section of pancreas was calculated using the following formula: Number of islets X 10⁶/total area of the section. Data were analyzed using a three factor analysis of variance. The main effects were treatment group (control vs. knockout), sex, and week of sacrifice (7 or 24). The model included the three two-way and the three-way interaction terms. Because the 24 week old male islet area data had an appreciably smaller standard deviation than all other factor combinations, the analysis was repeated using the log transformation. For adipose tissue, morphometric analysis was performed using the Image Pro Plus® analysis software. The number of adipocytes was determined in 6 randomly selected areas from the epididymal fat pads from 9-week old and 11.7-week old male mice.

Insulin and caspase-3 immunohistochemistry. Formalin-fixed paraffin-embedded sections of pancreas were deparaffinized, rehydrated, and incubated with 3% hydrogen peroxide for 10 mins in order to quench endogenous peroxidase activity. The sections were blocked with Dako® Protein Block (Dako Cytomation, Glostrup, Denmark, Cat. No. X0909) for 20 mins. Each of the steps following the application of the primary antibody were preceded by a rinse in wash buffer (Biogenex, San Ramon, Calif., Cat. No. HK583-5K). For insulin staining, the sections were then incubated with a guinea pig anti-insulin IgG (Dako Cytomation, Cat. No. A0564) diluted 1/1000 for 1 hour at room temperature. For caspase-3 staining, the sections were incubated with a rabbit IgG anti-cleaved caspase-3 (Asp175 clone) antibody (Cell Signaling Technology, Beverly, Mass.), diluted 1/150, for 1 hour at room temperature. The anti-insulin primary antibody was followed by a 45 min incubation with a biotin-labeled goat anti-guinea pig IgG (Vector Laboratories, Burlingame, Calif., Cat. No. BA-7000), diluted 1/150. The anti-caspase-3 primary antibody incubation was followed by a 45 min incubation with a biotin-labeled goat anti-rabbit IgG diluted at 1/150 (Vector Laboratories). Finally, the sections were incubated with the Vectastain® Elite ABC kit (Vector Laboratories, Cat. No. PK-6100) for 30 min, stained with a liquid DAB (Dako, Cat. No. K3468) for 5 min, according to the manufacturer's instructions, and counterstained with hematoxylin.

Micro CT scanning. Images were obtained using a commercially available micro CT system (MicroCAT®, ImTek Inc., Oak Ridge, Tenn.) with a high-resolution CCD/phosphor screen detector. The scanner consisted of a cylindrical diameter/long field view of 50 mm/50 mm with a spatial resolution of less than 50 μm. The X-ray source was biased at 40 KeV with the anode current set to 400 μA. Anesthetized mice were placed on a radio-transparent mouse bed in a supine position, caudal end closest to the micro CT with the rostral end held in place against an anesthesia delivery tube. The hind legs were moderately extended and held in place with clear tape to ensure a correct anatomical position (i.e. straight spine) and that the mouse position did not change once the scan procedure was initiated. An initial radiographic image was acquired at 90° to the plane of the mouse bed to allow correct positioning of the mouse by centering the scan acquisition area at the level of the iliac crest of each mouse.

Image reconstruction and analysis. Image reconstruction, whereby a micro CT scan of an individual mouse was manipulated to produce two-dimensional cross sectional images, was performed using the MicroCAT® Reconstruction, Visualization, and Analysis Software (ImTek Inc.) Two sets of reconstructed images per scan were generated for each mouse for the determination of individual fat depot mass. User-defined placement of reconstruction slices were placed relative to defined anatomical sites (i.e. vertebral segments). The first set of reconstructed images, consisting of six slices (intervertebral segments Lumbar 6-7 through Sacral 4-Caudal 1), provided a montage for the analysis of inguinal and epididymal adipose tissue depots. The second reconstruction set, consisting of nine slices (intervertebral and midvertebral landmarks from Lumbar 2-3 through Lumbar 6-7) was used to define retroperitoneal and mesenteric adipose tissue depot masses. Reconstructed bitmap images were converted to TIFF images and subsequently analyzed for fat depot mass using Scion Image for Windows (Scion Corporation, Frederick, Md.).

Liver glucose 6-phosphatase (G6P) and phosphoenolpyruvate carboxykinase (PEPCK) gene expression. Total RNA was purified from liver using the RNeasy® Mini Kit (Qiagen, Valencia, Calif.). RNAs were column treated with DNase prior to elution according to Qiagen's protocol. Superscript II® Reverse Transcriptase (Invitrogen) was used to make cDNA from total RNA alongside duplicate reactions run in the absence of reverse transcriptase (RT). G6P and PEPCK gene expression were quantitated by real time quantitative PCR (TaqMan®, Applied Biosystems, Foster City, Calif.) and normalized to the housekeeping gene 18S rRNA. ‘Minus RT’ reactions were also amplified in order to assess signal contribution by contaminating genomic DNA. TaqMan® PCR was performed by adding 25 ul 2× PCR Master Mix (Applied Biosystems), 5 μl of 2.5 μM probe (see below), 0.5 μl each of 30 μM forward and reverse primers (see below) and 14 μl water per well into a 96-well plate containing 1 ng of cDNA in a 5 μl volume (final volume of 50 μl). Amplification was performed on a 7700 Sequence Detection System (Applied Biosystems) according to a preset protocol: 50° C./2 min, 95°/10 min, followed by 40 cycles of 95° C./15 sec, 60° C./1 min. Data was analyzed using the 2^{-ΔΔCt} method (Livak et al., Methods 25:402-408, 2001) which was previously validated as demonstrating equivalent amplification efficiencies of the target genes G6P and PEPCK, and the reference gene 18S rRNA. Probes were custom made by Applied Biosystems. G6P: 6FAM-AGTCCC TCTGGCCATGCCATGG-TAMRA (SEQ ID NO: 5); PEPCK: 6FAM-AGGGCAAGATCATCATGCACGACC C-TAMRA (SEQ ID NO: 6). Primers were made by Invitrogen. G6P forward: CACCTGTGAGACCGGACCA (SEQ ID NO: 7), G6P reverse: GACCATAACATAGTATACACCTGCTGC (SEQ ID NO: 8); PEPCK forward: GACCATAACATAGTATACACCTGCTGC (SEQ ID NO: 9); PEPCK reverse: AGAAGGGTCGCATGGCAA (SEQ ID NO: 10). An 18S rRNA probe and primer kit was obtained from Applied Biosystems.

Liver glycogen synthase (GS) activity. Liver was polytronned in 10 volumes of GS Homogenization Buffer (10 mM TRIS-HCl, pH 7.4, 150 mM KF, 15 mM EDTA, 0.6 M sucrose, 1 mM PMSF, 1 mM benzamidine, 25 μg/ml leupeptin, and 50 mM 2ME) for 30 seconds on ice. Whole homogenates were assayed for protein using Bradford Reagent (BioRad, Hercules, Calif.) and IgG as standards. Glycogen synthase (GS) was measured according to the low/high G6P method (Guinovart et al., FEBS Letters 106:284-288, 1979), with a total reaction volume of 90 μl containing 100 uM UDP-[U-¹⁴C]-glucose (˜4.5 μCi/umol), 50 mM TRIS-HCl, pH 7.8, 25 mM KF, 12.5 mM EDTA, and 7 mg/ml rabbit liver glycogen. In addition, Glucose-6-phosphate (G6P) was used at 10 mM to measure total synthase activity (GS_{d}+GSᵢ) and at 0.1 mM to measure the phosphorylation-independent synthase activity (GSᵢ). 150 μg of liver homogenate was included in a 10 min incubation at 30° C. Reactions Were stopped by pipetting 65 μl onto 1.5 cm² pieces of Whatman 31 ET Chromatography paper which was dropped into 300 ml ice cold 50% ethanol and washed for 5 mins on a rotator. A second wash was done for 1 hour in 700 ml ice cold 50% ethanol followed by 1 min in 100 ml acetone. The squares were dried for 10 mins in a 60° C. oven, placed in scintillation fluid (Ready Safe™, Beckman Coulter, Fullerton, Calif.) and counted for ¹⁴C-glucose incorporation into glycogen on a liquid scintillation counter (LKB Wallac 1219 Rackbeta, Perkin Elmer Life Sciences, Boston, Mass.). The GS_{d} dpm were divided by the (GS_{d}+GSᵢ) activity and expressed as the GS activity ratio.

Isolated muscle glucose uptake assay. Soleus muscles were isolated from fed, male Akt2 KO and WT mice for the determination of glucose uptake in the absence or presence of insulin at the indicated concentrations as described previously (Etgen and Oldham, Metabolism 49: 684-688, 2000).

Results

Southern blot analysis of genomic DNA isolated from DBA/1lacJ ES cells confirmed the correct recombination (FIG. 2), and PCR genotyping analysis of F2 generation mice confirmed the generation of Akt2 KO mice (FIG. 3). Western blot analysis of protein lysates derived from brains isolated from Akt2^{+/+} and Akt2^{-/-} mice shows no detectable level of Akt2 protein in the Akt2 KO mice. Thus, the targeted disruption resulted in a functionally null allele.

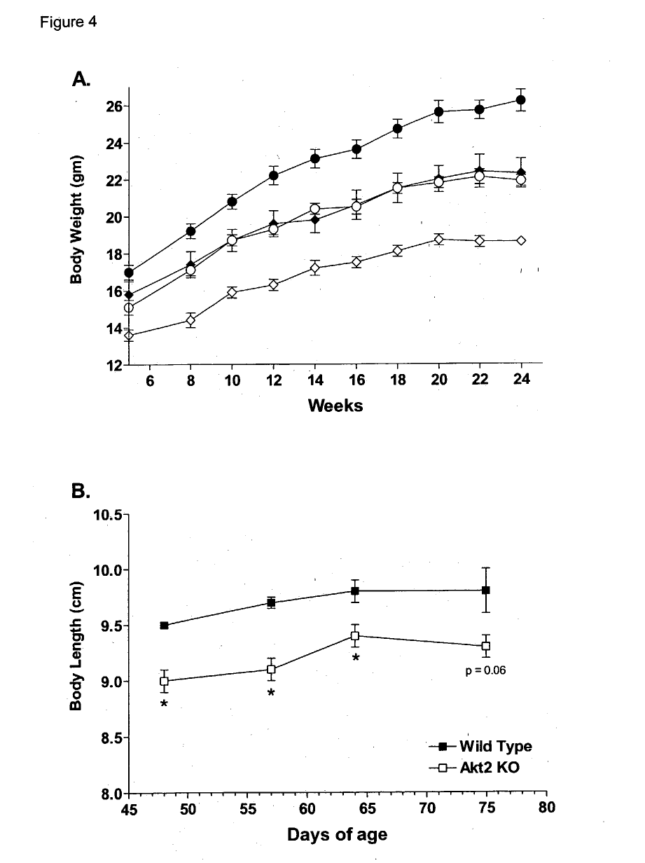

The Akt2^{-/-} mice (genetic background: DBA/1lacJ) were viable. Examination of 49 pups (10 litters) from matings between two Akt2^{±} heterozygous mice showed a Mendelian ratio among WT, heterozygous, and Akt2 KO mice. Both male and female Akt2 KO mice exhibited a growth deficiency (FIG. 4A). At 5 weeks of age, male and female Akt2 KO mice weighed, respectively, 11% and 14% less than control mice (p<0.01 for both sexes, n=9−13 for each group). This decrease in body weight persisted throughout life, averaging 13% and 16% for males and females, respectively, over 6 months. Akt2 KO mice also exhibited a modest but significant decrease in length as compared to control mice (FIG. 4B), with an average 5% difference evident from 6 to 11 weeks of age.

Comparison of body and selected organ weights of 7 week-old Akt2 KO and WT mice revealed several interesting differences (Table 1). While body weight was reduced by 16% in both males and females, the relative weight of brain and liver were increased by approximately 10% in Akt2 KO mice of both sexes (Table 1). In contrast, the relative amount of brown adipose tissue was reduced significantly in both Akt2 KO males (13%) and females (20%), whereas white adipose tissue, as assessed by Weight of the gonadal fat pad, was reduced by-more than 50% in KO females, but not significantly in KO males (Table 1).

TABLE 1
Fold change in weights in Akt2 KO relative to WT mice.

| | | Males | | | | |
|---|---|---|---|---|---|---|
| | | vs. | | | Females | |
| weight | | body | vs. | | vs. | vs. |
| (wt.) | absolute | wt. | brain wt. | absolute | body wt. | brain wt. |
| body | 0.84** | NA | NA | 0.84** | NA | NA |
| brain | 0.92** | 1.09* | NA | 0.94** | 1.11* | NA |
| BAT | 0.73** | 0.87* | 0.80** | 0.67** | 0.80* | 0.71** |
| WAT | 0.79 | 0.93 | 0.86 | 0.40** | 0.47** | 0.42** |
| kidney | 0.70** | 0.80** | 0.76** | 0.81** | 0.96 | 0.87* |
| liver | 0.95 | 1.12* | 1.04 | 0.93 | 1.09* | 0.98 |
| spleen | 0.71** | 0.85* | 0.78** | 0.87 | 1.03 | 0.93 |
| thymus | 0.76** | 0.91 | 0.83* | 0.94 | 0.91 | 0.82 |

*p < 0.05;
**p < 0.01

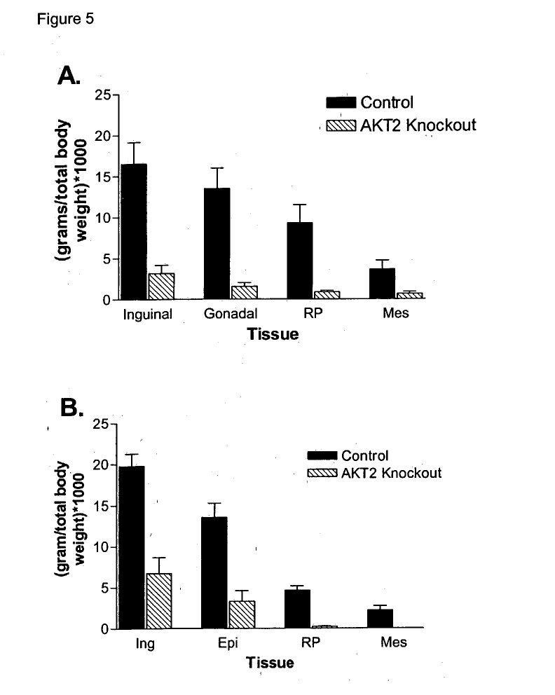

To further explore the observed differences in fat pad weights, microCT scanning was used to assess the size of multiple adipose depots. Akt2 KO animals (22 weeks of age) were found to exhibit significant lipoatrophy with all adipose depots in both males and females being dramatically reduced in size. The reduction in size of the fat depots in females was 80-90% (FIG. 5A). In males, the inguinal-subcutaneous and epididymal depots were reduced 65-75%, and the retroperitoneal and mesenteric depots were almost completely absent (FIG. 5B). The more significant reduction in size of the gonadal fat pads in both males and females at 22 weeks relative to 7 weeks indicates that loss of adipose tissue is progressive with age. Consistent with this, the weight of the epididymal fat pad was found to be similar in male WT and Akt2 KO mice at 9 weeks of age (0.21±0.04 vs 0.18+0.02 g, WT and Akt2 KO, respectively; p>0.05) but reduced by 24% in Akt2 KO mice at 11.7 weeks of age (0.21+0.11 vs 0.158+0.017, WT and Akt2 KO, respectively; p<0.05). Results of morphometric analysis of adipose tissue indicate that there is no significant difference in adipocyte cell size at 11.7 weeks, however, suggesting that the decrease in adipose mass is due to a decrease in cell number. As observed in other lipoatrophic syndromes plasma triglycerides were elevated by 60% in male Akt2 KO mice (248±21 mg/dl vs. 154±24 mg/dl, Akt2 KO and WT, respectively, p<0.05). The decrease in adipose tissue was also reflected in a decrease in plasma leptin concentration by 30% in male Akt2 KO mice (2.5±0.1 vs 3.5±0.5 ng/ml, for Akt2 KO and WT, respectively, p<0.05) and a trend towards lower leptin concentration in females (2.3±0.1 vs 3.1±0.4 ng/ml, for Akt2 KO and WT, respectively, p=0.09).

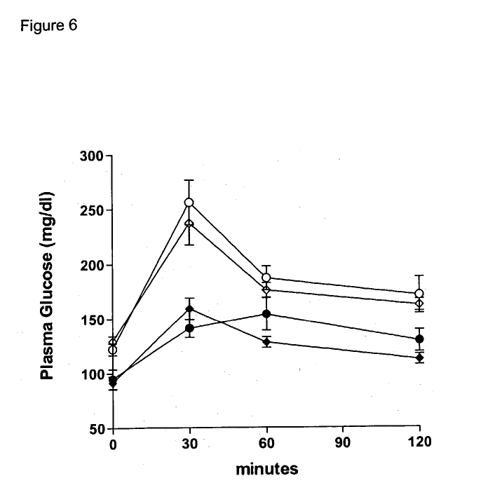

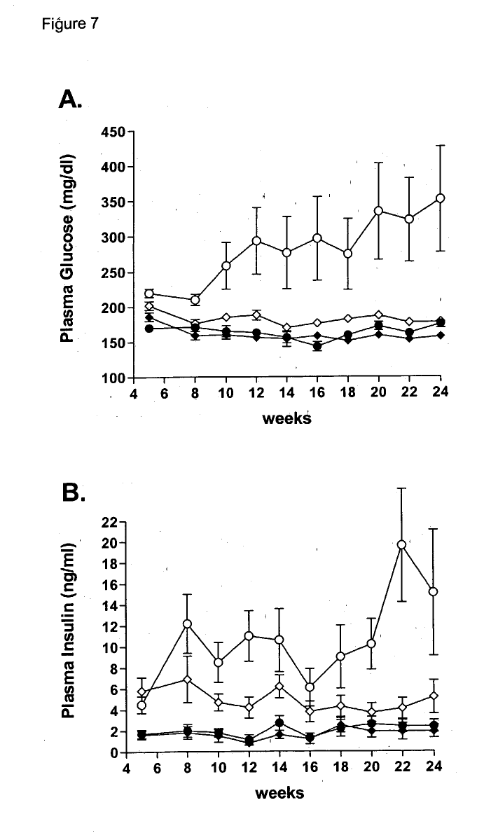

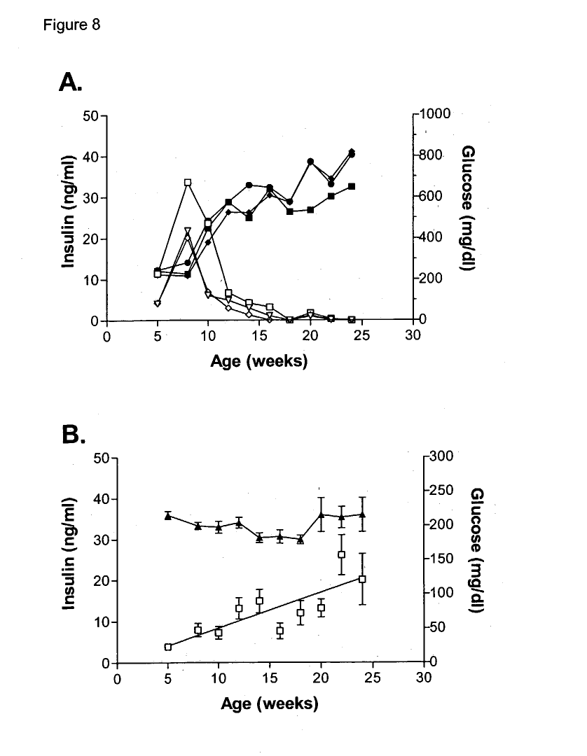

Both male and female Akt2 KO mice exhibited fasting hyperglycemia and glucose intolerance (FIG. 6). Fed hyperglycemia was observed in five week old male Akt2 KO mice (220 mg/dl for Akt2 KO vs 170 mg/dl for WT, p<0.001) and became more severe with age (FIG. 7A). Female mice exhibited a milder fed hyperglycemia that did not become significantly elevated until 10 weeks of age (FIG. 7A, 185 mg/dl for KO vs 160 mg/dl for WT, p=0.001) and remained stable until one year of age (182 mg/dl for KO vs 160 mg/dl for WT, p=0.024). Plasma insulin levels, however, were elevated in both males and females at all ages (FIG. 7B). In five-week-old male and female Akt2 KO mice, plasma insulin was elevated 2.6 and 3.6 fold, respectively (4.5 vs. 1.7 ng/ml for males and 5.8 vs 1.6 ng/ml for females, Akt2 KO and control, respectively). While insulin levels in female Akt2 KO mice remained stable for the duration of the six month long study (FIG. 7B), average insulin levels in male Akt2 KO mice increased further with age, suggestive of deteriorating insulin sensitivity. Furthermore, insulin levels in Akt2 KO males were more heterogeneous than in females, due in part to mice exhibiting two distinct patterns of insulinemia and glycemia over the six month period of observation. Three mice (25%) from this group exhibited a transient hyperinsulinemia that peaked at 8 weeks of age, followed by a decline in plasma insulin to undetectable levels by 15-18 weeks of age, suggestive of β cell failure (FIG. 8A). This was accompanied by progression to extreme hyperglycemia (FIG. 8A), with blood glucose values greater than 500 mg/dl evident by 12 weeks of age. The remaining mice exhibited a more stable and milder, albeit significant, hyperglycemia in the face of steadily increasing plasma insulin levels (FIG. 8B), consistent with deteriorating insulin sensitivity. Notably, in three separate cohorts of male Akt2 KO mice, a high percentage exhibited hypoinsulinemia with accompanying extreme hyperglycemia: 100% (n=6) of 8 month-old mice (FIG. 9), 80% (n=5) of 20 week-old mice, and 92% (n=25) of 7-8 month old mice. Thus, 75% of 48 male Akt2 KO mice progressed to this extreme diabetic phenotype between 5-8 months of age. The two groups of mice depicted in FIG. 8 may reflect temporal differences in the progression of the phenotype in the population, or, conversely, different susceptibilities to development of more extreme diabetes, possibly due to differences in prenatal or perinatal nutrition (Simmons et al., Diabetes 50:2279-2286, 2001; Garofano et al., FASEB J. 14:2611-2617, 2000).

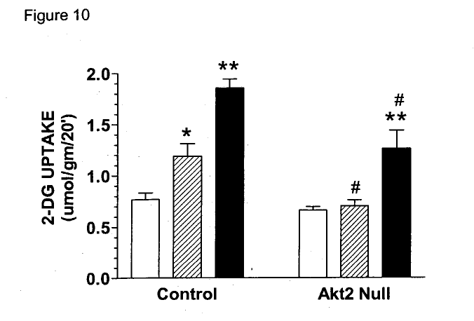

To determine whether impaired glucose disposal into skeletal muscle contributed to the hyperglycemia and insulin resistance of Akt2 KO mice, glucose uptake into isolated soleus muscles was examined (FIG. 10). No difference was observed in basal glucose uptake into muscles from control and Akt2 KO mice. However, submaximal (1 nM) insulin failed to increase glucose uptake above basal and uptake in response to maximal (100 nM) insulin was reduced in muscles from Akt2 KO mice (FIG. 10). Thus, the lack of Akt2 in skeletal muscle decreased both the insulin sensitivity and responsiveness of glucose transport.

Regulation of enzymes involved in glucose production and storage in liver was also abnormal in Akt2 KO mice. Expression of phosphoenolpyruvate carboxykinase (PEPCK), the rate-limiting enzyme of gluconeogenesis, was elevated prior to β cell failure in liver of diabetic 7-week old fed Akt2 KO mice by 1.9-fold relative to WT (p<0.05, Table 2). At 24 weeks of age, the level of PEPCK expression was consistent with the prevailing glycemia, being elevated 4.1-fold (p<0.01) in the subset of hypoinsulinemic, hyperglycemic (FIG. 8A) Akt2 KO mice, although it was not different between control and hyperinsulinemic (FIG. 8B) Akt2 KO mice which exhibited only mild hyperglycemia (Table 2). Glucose-6-phosphatase (G6Pase) expression was not elevated in Akt2 KO mice either at 7 or 20 weeks of age, except in the subset of hypoinsulinemic Akt2 KO mice, in which the level of G6Pase mRNA Was elevated 2.2-fold (p<0.05, Table 2). The proportion of liver glycogen synthase in the active state did not differ between fed control and Akt2 KO mice at 21 weeks of age (activity ratio, 0.055±0.009 vs 0.070±0.011, WT and Akt2 KO, respectively, p=0.312). However, total glycogen synthase activity measured in the presence of high (10 mM) glucose-6-phosphate was reduced by 46% in severely diabetic Akt2 KO mice (p<0.05), suggesting that the absolute amount of active glycogen synthase was decreased. 9-week old Akt2 KO mice that were still hyperinsulinemic, did not exhibit this decrease in total liver glycogen synthase activity.

TABLE 2
Expression of gluconeogenic enzymes in liver of male WT and Akt2
KO mice.

| | | | 24 weeks | | |
|---|---|---|---|---|---|
| | 7-weeks | | | KO | KO |
| Age | WT | KO | WT | Ins-H | Ins-L |
| Plasma Glucose | 233 ± 11 | 376 ± 19* | 175 ± 6 | 216 ± | 758 ± |
| (mg/dl) | | | | 25 | 55* |
| Plasma Insulin | 0.85 ± 0.1 | 54 ± 0.8* | 2.4 ± 0.6 | 20.1 ± | ND |
| (ng/ml) | | | | 6.3* | |
| PEPCK mRNA** | 2.8 | 5.3* | 4.4 | 2.3 | 18.4* |
| G6Pase mRNA** | 1.9 | 1.2 | 1.2 | 0.96 | 2.6* |

24-week old Akt2 KO mice are separated into hyperinsulinemic (Ins-H) and hypoinsulinemic (Ins-L) groups;
ND = not detectable;
*p < 0.05;
mRNA levels determined by quantitative RT-PCR as described in Methods and expressed as arbitrary units.

β cell area in Akt2 KO mice was compared to that of wild type mice at 7 and 24 weeks of age (Table 3). At both ages, no significant difference in either the number of islets/area or the percent islet area was observed in either male or female hyperinsulinemic Akt2 KO mice. However, pancreata from the hypoinsulinemic/hyperglycemic male Akt2 KO mice (FIGS. 8A and 9) were characterized by a variable (10-59%) decrease in the total number of islets as compared to their wild type controls. The vast majority of the remaining islets in this cohort of mice were distorted and contained only a few β cells scattered within the exocrine pancreas. The percentage of islets containing apoptotic cells, as indicated by caspase-3 staining, was increased in pancreata from 24-week old hypoinsulinemic male Akt2 KO mice (37% in KO mice vs <2% in WT or hyperinsulinemic Akt2 KO mice). Occasionally, inflammatory and necrotic cells, or mitotic figures, were observed within those remaining islets. The abnormal morphology of these islets did not allow for accurate measurement of their areas and prevented the inclusion of these animals in the statistical analysis.

TABLE 3
Morphometric analysis of β cell islets from 7 and 24 week old animals.

| Age | 7 weeks | | | | 24 weeks | | | |
|---|---|---|---|---|---|---|---|---|
| Sex | Male | | Female | | Male | | Female | |
| Genotype | +/+ | −/− | +/+ | −/− | +/+ | −/− | +/+ | −/− |
| # | 0.49 ± 0.16 | 0.43 ± 0.07 | 0.54 ± 0.11 | 0.57 ± 0.15 | 0.40 ± 0.07 | 0.36 ± 0.06 | 0.63 ± 0.17 | 0.65 ± 0.04 |
| Islet/μm² | | | | | | | | |
| % Islet | 1.45 ± 0.52 | 1.49 ± 0.51 | 1.83 ± 0.48 | 1.48 ± 0.56 | 0.69 ± 0.41 | 1.34 ± 0.22 | 1.97 ± 0.70 | 1.82 ± 0.50 |
| area | | | | | | | | |

3 animals from the Akt2 KO male group had a markedly decreased number of islets with atrophy of the remaining islets. The abnormal morphology of the islets did not permit accurate measurement of their surface area. Therefore, these outliers were not considered in the statistical analysis.
n = 5-9 animals per group.

Islets from wild type mice had abundant intracytoplasmic insulin staining in β cells. Insulin staining was diffuse and uniform except for cells at the periphery of the islets, which stained positive for glucagon. A decrease, predominantly in males, in the intensity of staining for insulin in the cytoplasm of β cells was observed in islets from Akt2 KO mice. The remaining β cells contained variable amounts of intracytoplasmic insulin interspersed with areas lacking immunohistochemical staining. The decreased staining for insulin in the Akt2^{-/-} mice was already evident at 7 weeks but did not progress in incidence or severity by 24 weeks of age. The islets from the hypoinsulinemic/hyperglycemic males were characterized by loss of normal islet architecture and severe loss of insulin staining with only occasional, weak intracytoplasmic staining of a few remaining cells, consistent with the very low levels of plasma insulin in these animals.

1. A genetically-modified mouse, wherein said mouse is homozygous for a modification resulting in a disrupted Akt2 gene in the genome of said mouse.

2. The mouse of claim 1, wherein said genetic modification results in lipoatrophy, insulin resistance, glucose intolerance, or reduced body weight.

3. A genetically-modified animal cell, wherein said cell is homozygous for a modification comprising a disrupted Akt2 gene.

4. The animal cell of claim 3, wherein said cell is an embryonic stem (ES) cell or an ES-like cell.

5. The animal cell of claim 3, wherein said cell is isolated from a genetically-modified mouse homozygous for a modification that results in a disrupted Akt2 gene.

6. The animal cell of claim 5, wherein said cell is an embryonic fibroblast, stem cell, neuron, skeletal or cardiac muscle cell, myoblast, brown or white adipocyte, hepatocyte, or pancreatic β cell.

7. The animal cell of claim 3, wherein said cell is murine.

8. The animal cell of claim 3, wherein said cell is human.

9. A method of identifying a therapeutic agent for diabetes, said method comprising administering an agent to a genetically-modified mouse homozygous for a disrupted Akt2 gene and assessing a diabetic phenotype in said mammal, wherein said agent is identified as a treatment for diabetes if said mammal demonstrates an improvement in said diabetic phenotype.

10. The method of claim 9, wherein said improved diabetic phenotype is reduced plasma glucose or increased insulin sensitivity.

11. A method of identifying a gene that demonstrates modified expression as a result of reduced Akt2 activity in an animal cell, said method comprising assessing the expression profile of at least one gene other than Akt2 of an animal cell homozygous for a genetic modification that disrupts an Akt2 gene, and comparing said profile to that from a wild type cell.

12. A method of identifying a protein that demonstrates a modified level or post-translational processing as a result of reduced Akt2 activity in an animal cell comprising comparing the level or post-translational characteristics of the protein in an animal cell homozygous for a genetic modification that disrupts the Akt2 gene to the level or post-translational characteristics of the protein in an appropriate wild-type control.

13. A method of identifying a biological characteristic associated with reduction or elimination of Akt2 activity comprising comparing a biological characteristic of a genetically-modified mouse homozygous for a genetic modification that disrupts the Akt2 gene, or a genetically-modified animal cell homozygous for a genetic modification that disrupts the Akt2 gene, to the characteristic of the appropriate wild-type control.

14. A method of treating a patient for diabetes or growth restriction, said method comprising administering an agent that increases Akt2 activity in an amount sufficient to improve the diabetic or restricted growth condition.
